(19) United States
(12) Patent Application Publication
(10) Pub. No.: US 2012/0172292 A1
(43) Pub. Date: Jul. 5, 2012

(54) METHOD FOR PROTECTION OF ANTIMICROBIAL AND ANTICANCER DRUGS FROM INACTIVATION BY NITRIC OXIDE

(75) Inventors: Evgeny A. Nudler, New York, NY (US); Ivan Gusarov, Springfield, NJ (US)

(73) Assignee: NEW YORK UNIVERSITY, New York, NY (US)

(21) Appl. No.: 13/395,154

(22) PCT Filed: Sep. 10, 2010
(86) PCT No.: PCT/US10/48469

§ 371 Date: Mar. 9, 2012

Related U.S. Application Data
(60) Provisional application No. 61/241,238, filed on Sep. 10, 2009.

(51) Int. Cl. A61K 31/47; A61K 31/473; A61K 31/5415; A61K 31/65; A61K 31/7036; A61K 31/546; A61K 31/545; A61K 31/431; A61K 31/5365; A61K 31/495; A61P 31/00; A61P 33/02; A61P 35/00; A61K 31/704; A61K 31/4745; A61K 31/337; A61K 38/02; A61K 31/7048

(52) U.S. Cl. 514/4.4; 514/311; 514/27; 514/297; 514/225.8; 514/226.2; 514/154; 514/40; 514/203; 514/207; 514/209; 514/202; 514/196; 514/210.08; 514/255.02; 514/2.3; 514/19.3; 514/34; 514/283; 514/449; 514/292

(57) ABSTRACT

This invention discloses a method for enhancing the efficacy of antimicrobial, anti-protozoa and anti-cancer treatments by co-administering an inhibitor of endogenous NO production and/or NO scavenger.

### REFERENCE TO RELATED APPLICATIONS

The present application claims the benefit of U.S. Provisional Application No. 61/241,238, filed Sep. 10, 2009, which is incorporated herein by reference in its entirety.

### STATEMENT REGARDING FEDERALLY SPONSORED RESEARCH

Research and development leading to certain aspects of the present invention were supported, in part, by a grant from NIH AI60762 and NIH Director's Pioneer Award. Accordingly, the U.S. government may have certain rights in the invention.

### TECHNICAL FIELD OF THE INVENTION

This invention relates to a method for enhancing the efficacy of antimicrobial, anti-protozoa and anti-cancer treatments by co-administering an inhibitor of endogenous NO production and/or NO scavenger.

### BACKGROUND OF THE INVENTION

Bacterial NO-synthases (bNOS) are present in many Gram-positive species and have been demonstrated to synthesize NO from arginine in vitro and in vivo. However, the physiological role of bNOS remains largely unknown. bNOS and its eukaryotic counterparts, which produce NO by catalyzing the oxidation of L-arginine to L-citrulline, are structurally and mechanistically related (1-3). Although bNOS lacks the essential reductase domain, it uses available cellular reductases to generate NO in vivo (4). Previously, it has been demonstrated that bNOS protects bacteria against oxidative stress (5, 6). This function of bNOS was found to be essential for some pathogenic organisms. For example, the survival of Bacillus anthracis (B.anthracis) in macrophages strictly depends on bNOS activity, which is an important virulence factor that protects this pathogen from immunological oxidative bursts (6). bNOS has also been shown to function during Streptomyces turgidiscabies infection of plants (7). bNOS genes are also present in the genomes of numerous nonpathogenic soil bacteria (4).

In mammals, nitric oxide synthase (NOS) exists in two major forms, constitutive and inducible. Reviewed in Rodeberg et al., Am. J. Surg. 170:292-303 (1995) and Bredt and Snyder, Ann. Rev. Biochem. 63:175-95 (1994)). Under physiological conditions, a low output of NO is produced by the constitutive, calcium-dependent NOS isoform (cNOS) present in numerous cells, including endothelium and neurons. This low level of NO is involved in a variety of regulatory processes, e.g., blood vessel homeostasis, neuronal communication and immune system function. On the other hand, under pathophysiological conditions, a high output of NO is produced by the inducible, calcium-independent NOS isoform (iNOS) which is expressed in numerous cell types, including endothelial cells, smooth muscle cells and macrophages. These high levels of NO have been shown to contribute to inflammation-related tissue damage, neuronal pathology, N-nitrosamine-induced carcinogenesis and mutations in human cells and bacteria via deamination reaction with DNA. NO can therefore be seen to be a mixed blessing, being very desirable when present in small amounts, while potentially being highly detrimental when produced in excessive quantities.

Despite the phenomenal success of antibiotics, infectious diseases remain the second leading cause of death worldwide. About two million Americans are infected in hospitals each year (90,000 of them die of it), and more than half of these infections resist at least one antibiotic. Most alarmingly, pathogens become fully resistant to the last resort antibiotics, such as vancomycin. The emergence of multidrug-resistant bacteria has created a situation in which there are few or no options for treating certain infections. Natural antibiotics and their derivatives are intrinsically prone to become obsolete because of preexisting genes that render pathogens resistant to them. Bacterial species share these genes thus rapidly spreading resistance from hospitals and farms to surrounding communities.

Approximately 40% of the world population lives in areas with the risk of malaria. Each year, 300-500 million people suffer from acute malaria, and 0.5-2.5 million die from the disease. Although malaria has been widely eradicated in many parts of the world, the global number of cases continues to rise. The most important reason for this alarming situation is the rapid spread of malaria parasites that are resistant to antimalarial drugs, especially chloroquine, which is by far the most frequently used.

Thus, there is a great need to enhance the efficacy of antimicrobial and anti-malarial treatments.

### SUMMARY OF THE INVENTION

The present invention fulfills these and other related needs by providing a novel method for enhancing the efficacy of antimicrobial, anti-protozoa and anti-cancer treatments by co-administering an inhibitor of endogenous NO production and/or NO scavenger.

In one object, the present invention provides a method for enhancing efficacy of an antimicrobial, anti-protozoa or anti-cancer treatment in a subject, wherein said treatment comprises administering to the subject a compound which becomes inactivated by NO or natural products of NO oxidation in vivo or becomes less effective due to NO action, said method comprising co-administering said compound with an inhibitor of endogenous NO production and/or NO scavenger.

In one specific embodiment, the compound which becomes inactivated by NO or natural products of NO oxidation in vivo or becomes less effective due to NO action and the inhibitor of endogenous NO production or NO scavenger are administered simultaneously. In another specific embodiment, the compound which becomes inactivated by NO or natural products of NO oxidation in vivo or becomes less effective due to NO action and the inhibitor of endogenous NO production or NO scavenger are administered sequentially. In yet another embodiment, the compound which becomes inactivated by NO or natural products of NO oxidation in vivo or becomes less effective due to NO action and the inhibitor of endogenous NO production or NO scavenger are administered in the same composition. In a separate embodiment, the compound which becomes inactivated by NO or natural products of NO oxidation in vivo or becomes less effective due to NO action and the inhibitor of endogenous NO production or NO scavenger are administered in different compositions.

In one embodiment, the inhibitor of endogenous NO production is selected from the group consisting of L-arginine, N G -monomethyl-L-arginine (NMMA), N G -nitro-L-arginine methyl ester (NAME), N G -nitro-L-arginine (NNA), N G -amino-L-arginine (NAA), N G,N G-dimethylarginine (asymmetric dimethylarginine, called ADMA), L-Thiocitrulline, S-methyl-L-Thiocitrulline, diphenyleneiodonium chloride, 2-(4-carboxyphenyl)-4,4,5,5-tetramethylimidazoline-1-oxy 3-oxide, 7-nitroindazole, N(5)-(1-iminoethyl)-L-ornithine, aminoguanidine, canavanine, ebselen, S-methyl-L-citrulline, S-methylisourea, and 2-mercaptoethylguanidine. In another embodiment, the inhibitor of endogenous NO production is an iNOS-specific inhibitor.

In one embodiment, the NO scavenger is selected from the group consisting of non-heme iron-containing peptides, non-heme iron-containing proteins, porphyrins, metalloporphyrins, dithiocarbamates, dimercaptosuccinic acid, phenanthroline, desferrioxamine, pyridoxal isonicotinoyl hydrazone (PIH), 1,2-dimethyl-3hydroxypyrid-4-one (L1), [+] 1,2-bis (3,5-dioxopiperazine-1yl)propane (ICRF-187), and 2-(4-carboxyphenyl)-4,5-dihydro-4,4,5,5-tetramethyl-1H-imidazolyl-1-oxy-3-oxide (Carboxy-PTIO). In another embodiment, the NO scavenger is a perfluorocarbon emulsion.

In one embodiment, the compound which becomes inactivated by NO or natural products of NO oxidation in vivo or becomes less effective due to NO action is an antimicrobial compound selected from the compounds disclosed in FIGS. 1A and 6A-B and Table 1, below. In a specific embodiment, the antimicrobial compound is selected from the group consisting of 5-Chloro-7-iodo-8-hydroxyquinoline, 8-Hydroxyquinoline, 8-Hydroxy-5-nitroquinoline, Novobiocin, Acriflavine, 9-Aminoacridine, Prochlorperazine, Chlorpromazine, Prochlorperazine, Penimepicycline, Sisomicin, Gentamicin, Cephaloridine, 7-Aminocephalosporanic acid, Cefotaxime, Cefuroxime, Ampicillin, Moxalactam, 6-Aminopenicillanic acid, Amoxicillin, Azlocillin, Proflavine, Panflavine, Planacrine, Gonoflavin, Trypaflavin, Diflavine, Flavicid, Ethacridine (Rivanol), Aminacrine, 3-Amino-10-methyl-6-haloacridinium, 3-Nitro-9-aminoacridine, 9-Amino-2,3-dimethoxy-6-nitroacridine-10-oxides, and Salacrin.

In another embodiment, the compound which becomes inactivated by NO or natural products of NO oxidation in vivo or becomes less effective due to NO action is an anti-cancer compound selected from the compounds disclosed in FIG. 12B and Tables 2-3, below. In a specific embodiment, the anti-cancer compound is an acridine derivative selected from the group consisting of topoisomerase inhibitors (e.g., m-AMSA Amsacrine, AMSA-carboximide, Asulacrine (CI-921), AMCA, m-AMCA, amino-DACA, As-DACA, and NETGA), acridine-platinum conjugates, acridine-alkylating agents, telomerase inhibitors, and DNA crosslinking agents (e.g., Ledakrine). In another specific embodiment, the anti-cancer compound is selected from the group consisting of Doxorubicin, Daunorubicin, Mitoxantrone, Actinomycin D, Mithramycin A, Mitomycin C, Bleomycin, Vincristine, Vinorelbine, Paclitaxel, Docetaxel, Irinotecan, Topotecan, and Fumitremorgin C.

In yet another embodiment, the compound which becomes inactivated by NO or natural products of NO oxidation in vivo or becomes less effective due to NO action is an anti-protozoa compound selected from the compounds disclosed in FIG. 12B and Table 4, below. In a specific embodiment, the anti-protozoa compound is Pyronaridine or Amodiaquine.

In one embodiment, the treatment is directed against an infection by S.aureus or B. anthracis. In another embodiment, the treatment is directed against an infection causing pneumonia or endocarditis (e.g., S.aureus infection).

In another embodiment, the treatment is directed against a malarial infection.

In a second object, the present invention provides a method for decreasing an effective concentration of a drug used in an antibacterial, anti-protozoa or chemotherapeutic treatment, wherein said drug becomes inactivated by NO or natural products of NO oxidation in vivo or becomes less effective due to NO action, said method comprising co-administering said drug with an inhibitor of endogenous NO production and/or NO scavenger.

In one specific embodiment, the drug and the inhibitor of endogenous NO production or NO scavenger are administered simultaneously. In another embodiment, the drug and the inhibitor of endogenous NO production or NO scavenger are administered sequentially. In yet another embodiment, the drug and the inhibitor of endogenous NO production or NO scavenger are administered in the same composition. In a separate embodiment, the drug and the inhibitor of endogenous NO production or NO scavenger are administered in different compositions.

In one embodiment, the inhibitor of endogenous NO production is selected from the group consisting of L-arginine, N G -monomethyl-L-arginine (NMMA), N G -nitro-L-arginine methyl ester (NAME), N G -nitro-L-arginine (NNA), N G -amino-L-arginine (NAA), N G,N G -dimethylarginine (asymmetric dimethylarginine, called ADMA), L-Thiocitrulline, S-methyl-L-Thiocitrulline, diphenyleneiodonium chloride, 2-(4-carboxyphenyl)-4,4,5,5-tetramethylimidazoline-1-oxy 3-oxide, 7-nitroindazole, N(5)-(1-iminoethyl)-L-ornithine, aminoguanidine, canavanine, ebselen, S-methyl-L-citrulline, S-methylisourea, and 2-mercaptoethylguanidine. In another embodiment, the inhibitor of endogenous NO production is an iNOS-specific inhibitor.

In a specific embodiment, the NO scavenger is selected from the group consisting of non-heme iron-containing peptides, non-heme iron-containing proteins, porphyrins, metalloporphyrins, dithiocarbamates, dimercaptosuccinic acid, phenanthroline, desferrioxamine, pyridoxal isonicotinoyl hydrazone (PIH), 1,2-dimethyl-3hydroxypyrid-4-one (L1), [+] 1,2-bis (3,5-dioxopiperazine-1yl)propane (ICRF-187), and 2-(4-carboxyphenyl)-4,5-dihydro-4,4,5,5-tetramethyl-1H-imidazolyl-1-oxy-3-oxide (Carboxy-PTIO). In another embodiment, the NO scavenger is a perfluorocarbon emulsion.

In one embodiment, the drug is selected from the compounds disclosed in FIGS. 1A, 6A-B, 12B and Tables 1-4, below. In a specific embodiment, the drug is an antimicrobial compound selected from the group consisting of 5-Chloro-7-iodo-8-hydroxyquinoline, 8-Hydroxyquinoline, 8-Hydroxy-5-nitroquinoline, Novobiocin, Acriflavine, 9-Aminoacridine, Prochlorperazine, Chlorpromazine, Prochlorperazine, Penimepicycline, Sisomicin, Gentamicin, Cephaloridine, 7-Aminocephalosporanic acid, Cefotaxime, Cefuroxime, Ampicillin, Moxalactam, 6-Aminopenicillanic acid, Amoxicillin, Azlocillin, Proflavine, Panflavine, Planacrine, Gonoflavin, Trypaflavin, Diflavine, Flavicid, Ethacridine (Rivanol), Aminacrine, 3-Amino-10-methyl-6-haloacridinium, 3-Nitro-9-aminoacridine, 9-Amino-2,3-dimethoxy-6-nitroacridine-10-oxides, and Salacrin. In another specific embodiment, the drug is an anti-cancer acridine derivative selected from the group consisting of topoisomerase inhibitors (e.g., m-AMSA Amsacrine, AMSA-carboximide, Asulacrine (CI-921), AMCA, m-AMCA, amino-DACA, As-DACA, and NETGA), acridine-platinum conjugates, acridine-alkylating agents, telomerase inhibitors, and DNA crosslinking agents (e.g., Ledakrine). In yet another specific embodiment, the drug is an anti-cancer compound selected from the group consisting of Doxorubicin, Daunorubicin, Mitoxantrone, Actinomycin D, Mithramycin A, Mitomycin C, Bleomycin, Vincristine, Vinorelbine, Paclitaxel, Docetaxel, Irinotecan, Topotecan, and Fumitremorgin C. In a further specific embodiment, the drug is an anti-protozoa compound Pyronaridine or Amodiaquine.

These and other aspects of the present invention will become apparent upon reference to the following detailed description and attached drawings. All publications, patents, and patent applications cited herein, whether supra or infra, are hereby incorporated by reference in their entirety.

### BRIEF DESCRIPTION OF THE FIGURES

FIG. 1. bNOS protection against a wide spectrum of antimicrobials. (A) A representative list of chemicals from the Phenotype MicroArray screen that preferentially inhibit the growth of nos deficient B.subtilis. Negative numbers indicate the relative growth inhibition (as provided in FIG. 6A-B) of the Δnos strain compared to that of the wt strain (B) The integrated mechanism of NO-mediated defense against bactericidal antibiotics and toxins. Despite different primary targets, many bactericidal antibiotics kill bacteria by inducing oxidative stress (11). Here it is shown that NO protection occurs via two major pathways: (1) direct detoxification of a toxic compound (e.g., acriflavine and AMSAcrine) and (2) alleviation of the oxidative stress caused by many antimicrobials. The ability of NO to alleviate the oxidative stress is achieved by two mechanisms: (i) rapid protection via Fenton reaction inhibition and direct catalase (KatA) activation (5, 6) and (ii) induction of superoxide dismutase (SodA) expression. bNOS activity is stimulated by antibiotics, thereby ensuring the specific defense response.

FIG. 2. Mechanisms of bNOS protection against acriflavine. (A) Proposed chemistry of NO-mediated detoxification of ACR. (B) bNOS-dependent growth of B.anthracis in the presence of ACR. B.anthracis Sterne and Δnos overnight cultures were diluted into fresh LB media containing 8 μg/ml ACR. Cells were grown at 37° C. with aeration. (C) Changes in absorbance spectra of ACR upon interaction with NO. (D) Exogenous NO protects B.subtilis against ACR, but not against acridine orange (AO). Conditions were as in (B), except that 10 μg/ml ACR or AO were used. 30 μM of the NO donor, MAHMA, was premixed with ACR or AO in LB media and incubated for 5 min prior to inoculation with bacteria. Data are shown as the mean±SE from three experiments. (E) NO-dependent degradation of ACR in vivo. The plot shows intracellular ACR concentration normalized per mg of total protein of E. coli harboring either empty vector or pNOS_{Ban}. Cells were induced by arabinose at OD₆₀₀˜0.3 for 30 min, followed by ACR (2 μg/ml) and arginine (5 mM) addition. Cells were collected, pelleted, and lysed. The clarified supernatant was used to measure OD₄₅₀ and protein concentration. A standard curve was generated to convert OD₄₅₀ to ACR concentration. Data are shown as the mean±SE from three experiments. (F) NO and the iron chelator bipyridyl act in the same pathway to protect cells against ACR. B.subtilis were pretreated with 0.5 mM bipyridyl (By) or 100 μM NO donor (NO) for 3 min, followed by challenge with 30 μg/ml ACR. The percentage of surviving cells was determined by colony formation, and is shown as the mean±SD from three experiments.

FIG. 3. NO-mediated defense against P.aeruginosa and its mechanism. (A) B.subtilis-generated NO allows growth in the presence of PYO. Overnight cultures of B.subtilis 6051 and Δnos strains were diluted in fresh LB medium for 1 hour, followed by addition of PYO to 25 μM (time 0). An NO donor (green triangles) or glucose (blue triangles) was added to aliquots of the Δnos cells 1 h after PYO challenge. Data are shown as the mean±SE from three experiments. The insert shows tubes with wt (left) and Δnos (right) cultures after a 4 h incubation with PYO. (B) Deletion of the nos gene sensitizes B.anthracis to PYO. Overnight cultures of B.anthracis Sterne (squares) and Δnos (circles) strains were diluted into fresh BHI medium supplemented with 100 μM PYO. The insert shows tubes with the Sterne (left) and Δnos (right) strains after an 8 h incubation with PYO. (C) SodA is critical for bacterial defense against PYO. Experimental conditions were as in (A), except that wt B. subtilis 168 was used as a background strain for all the mutants. Values are the means and ±SD from three independent experiments. (D) Chemical structure of the PYO toxin. (E) B.subtilis growth on PYO plates as a function of bNOS activity. A paper disk saturated with 10 mM PYO was placed atop the bacterial lawn. “baNOS” stands for B.subtilis expressing nos from B.anthracis. To induce nos expression and NO synthesis 2% arabinose and 1 mM Arg were added. Lysis zone borders are marked with dashed lines. (F) bNOS controls SodA expression. The pMutin vector was used to place the lacZ reporter under a chromosomal copy of the sodA promoter in B.subtilis 168 (wt) and Δnos strains. Overnight cultures were diluted in fresh LB and sampled to measure the growth (OD₆₀₀, open symbols) and β-galactosidase activity (filled symbols). SodA promoter induction was calculated based on the change in Miller units. Mean ±SD from three experiments. (G) Endogenous NO protects B.subtilis and B.anthracis from P.aeruginosa. 5 μl of a P.aeruginosa PA-14 overnight culture was placed atop the Bacilli lawns on P agar plates. Lysis zone borders are marked with dashed lines.

FIG. 4. The mechanism bNOS protection against cefuroxime. (A) Chemical structure of cefuroxime (CEF). (B) bNOS-dependent growth of S.aureus in the presence of CEF. Overnight cultures of S.aureus 4220 and its Δnos derivative were diluted into fresh LB media containing 0.4 μg/ml CEF. Cells were grown in triplicate at 37° C. with aeration using a Bioscreen C automated growth analysis system. (C) nos deletion renders B.subtilis more sensitive to cefuroxime. Overnight cultures of B.subtilis 6051 and Δnos strains were diluted into fresh LB medium and grown to OD.₆₀₀˜1.0, followed by the addition of 25 μg/ml cefuroxime (time 0). Aliquots were plated on LB agar and CFU counted the next day. Values are the means and ±SD from three independent experiments. (D) Stimulation of bNOS activity by antibiotic treatment. Conditions were the same as in (C). The graph demonstrates the changes in the total nitrite/nitrate concentration in wt and Δnos cultures before and after challenge with 50 μg/ml CEF. (E) NO protects B.subtilis against ROS-mediated CEF toxicity. Conditions were the same as in (C). Cells were pretreated with 0.5 mM bipyridyl (an iron chelator) or 100 μM NO donor or 150 mM thiourea (a ROS scavenger) for 3 min, followed a challenge with 50 μg/ml CEF. The percentage of surviving cells was determined by colony formation and is shown as the mean±SD from four experiments.

FIG. 5. NO-mediated detoxification in mammalian cells. (A) Chemical structure of AMSAcrine (AMSA). The amino group that can be attacked by NO⁺ (FIG. 12A) is indicated by an arrow. (B) NO drastically increases cell resistance to AMSAcrine. HepG2 human hepatocytes were challenged with AMSA alone or with AMSA premixed with NO. Where indicated, cells were pretreated with the NOS inhibitor L-NAME. The experiment was preformed in triplicate and the mean is shown. (C) Changes in absorbance spectra of AMSAcrine upon interaction with NO. (D) iNOS-dependent AMSAcrine degradation in vivo. Conditions were as in (B) except that media without phenol red was used. Aliquots of supernatants were collected immediately, 1 and 2 days after AMSAcrine addition to the HepG2 cells and the OD₄₃₅ was measured. The OD₄₃₅ was converted to AMSAcrine concentration according to a standard curve. The experiment was preformed in quadruplicate and the mean is shown.

FIG. 6. The results of Phenotype MicroArray. (A) The growth curves for B.subtilis wt are shown in grey, for Δnos in dotted grey, and overlay is shown in white. Data are shown as the means from two experiments. (B) The relative values of growth inhibition (negative numbers) are presented in the table.

FIG. 7. (A) bNOS increases the resistance of B.subtilis to ACR. B.subtilis 168 and Δnos overnight cultures were diluted into fresh LB media containing 8 μg/ml ACR. The cells were grown at 37° C. with aeration. Data are shown as the means±SE from four experiments. (B) Exogenous NO protects S.aureus against ACR. An overnight culture of S.aureus RN6734 was diluted into fresh LB media containing ether 20 μg/ml ACR (circles) or 100 μM of the NO donor MAHMA premixed with ACR in LB media for 5 min prior to inoculation with bacteria (triangles). The cells were grown at 37° C. with aeration.

FIG. 8. (A) bNOS increases the resistance of S.aureus to PYO. The plot shows the growth curves obtained on the Bioscreen C automated growth analysis system (Oy Growth Curves Ab Ltd). Overnight cultures of S.aureus 4220 (wt) and Δnos strains were diluted into saline to reach the OD600=0.1. The resulted stock was used as 10× to inoculate a 100 wells microplates filled with LB or LB with PYO. In all wells LB was supplemented with 50 μg/ml phenylalanine Plates were incubated with shaking at 37° C. The experiment was performed in triplicates and the means±SE is presented on the plot. (B) bNOS expression increases the resistance of B.subtilis to PYO. Overnight cultures of control and baNOS strains were diluted into fresh LB media supplemented with 2% arabinose and 1 mM Arg. The cells were grown for 1 hour followed by addition of 100 μM PYO (time 0). baNOS is a B.subtilis strain expressing B.anthracis NOS. Arabinose was used to induce nos expression.

FIG. 9. PYO detoxification by NO (A) NO does not detoxify PYO directly. Overnight cultures of B.subtilis 6051 and Δnos strains were diluted into fresh LB medium and grown for 1 hour at 37° C. with aeration. Then 25 μM PYO was added to the wt strain (inverted triangles, time 0). The B.subtilis Δnos strain was split in three tubes. PYO was added to the first tube (squares). The NO donor was added to the second tube 40 minutes after PYO addition (circles), and the 30 min preincubated mixture of PYO and the NO donor was added to the third tube (triangles). (B) PYO production by P.aeruginosa is responsible for the more efficient killing of the B.subtilis nos deficient mutant. Conditions were the same as in FIG. 3E, except that P.aeruginosa PA-01 and the corresponding ΔphzA1 mutant strains deficient in PYO production were used for co-culture experiments.

FIG. 10. NO does not detoxify CEF directly. Overnight cultures of B.subtilis 168 strain was diluted into fresh LB medium and grown till OD.600˜1.0 followed by the addition of 50 μg/m1 (110 μM) CEF (squares) or same concentration of CEF pretreated with NO (circles). Aliquots were plated on LB agar and CFU counted the next day. Values are the means and SD from three independent experiments. Either 100 μM NO donor MAHMA NONOate (A), or 200 μM acidified nitrite (B) was used for CEF pretreatment. In case of acidified nitrite 200 μM NaNO₂ was added to 110 μM CEF followed by 10 mM HCl. After 1 hour of incubation 10 mM NaOH was added to neutralize acidity. In control (B) CEF was incubated under the same conditions in 10 mM HCl followed by quenching with NaOH.

FIG. 11. Stimulation of endogenous NO production by CEF antibiotic. (A) The Cu(II)-based NO-detecting probe (CuFL) and fluorescent chemistry of NO detection (M. H. Lim, D. Xu, S. J. Lippard, Nat Chem Biol 2, 375-80 (July, 2006)). (B) The increase of NO production in vivo in response to CEF challenge. Cells were grown in LB to OD600˜0.9 followed by addition of freshly prepared CuFL (20 μM) and CEF (20 μg). Fluorescence was measured after 18 hours of incubation in total culture using real-time fluorometer (PerkinElmer LS-55). Data was normalized to Δnos control cells that were grown, treated with CuFL and CEF, and processed the same way. Lower panels show representative fluorescent images of non-treated B.anthracis Sterne (left) and treated with CEF (right) after incubation with CuFL.

FIG. 12. (A) A mechanism of anticancer and anti-protozoa drug inactivation by NOx. Products of NO auto-oxidations (e.g., N₂O₃) can readily nitrosate aromatic amines to form highly reactive diazonium cation intermediates, which are rapidly hydrolyzed or cross-linked to each other to produce less toxic compounds. (B) Examples of Clinically approved anticancer and anti-protozoa drugs that can be inactivated by NO+. The common mechanism of inactivation is described in panel (A).

### DETAILED DESCRIPTION OF THE INVENTION

The present invention is based on an unexpected discovery that endogenous NO compromises the activity of numerous antimicrobials and toxins and thus reduces the efficacy of antimicrobial, anti-protozoa and anti-cancer treatments using these agents. Specifically, as disclosed in the Examples, below, the present inventors have discovered that NO generated by bacterial NO-synthases (bNOS) present in many Gram-positive species increases the resistance of bacteria to a broad spectrum of antibiotics. NO-mediated resistance is achieved through both chemical modification of toxic compounds and alleviation of the oxidative stress imposed by many antibiotics. NO-mediated detoxification occurs in mammalian cells as well.

The present invention thus provides a method for enhancing efficacy of antimicrobial, anti-protozoa and anti-cancer treatments in a subject, wherein said treatments comprise administering to the subject compounds which become inactivated by NO and/or natural products of NO oxidation in vivo and/or become less effective due to NO action (e.g., because NO protects against oxidative stress and those compounds exert their toxicity via oxidative stress), said method comprising co-administering said treatments with an inhibitor of endogenous NO production and/or NO scavenger. Any co-administration regimen is encompassed by the present invention. For example, (i) compounds which become inactivated by NO and/or natural products of NO oxidation in vivo and/or become less effective due to NO action and (ii) an inhibitor of endogenous NO production and/or NO scavenger can be administered simultaneously or sequentially (i.e., before or after) and can be administered either in the same or in different compositions.

Specific non-limiting examples of useful inhibitors of endogenous NO production include L-arginine, N G -monomethyl-L-arginine (NMMA), N G -nitro-L-arginine methyl ester (NAME), N G -nitro-L-arginine (NNA), N G -amino-L-arginine (NAA), N G,N G -dimethylarginine (asymmetric dimethylarginine, called ADMA), L-Thiocitrulline, S-methyl-L-Thiocitrulline, diphenyleneiodonium chloride, 2-(4-carboxyphenyl)-4,4,5,5-tetramethylimidazoline-1-oxy 3-oxide, 7-nitroindazole, N(5)-(1-iminoethyl)-L-ornithine, aminoguanidine, canavanine, ebselen, S-methyl-L-citrulline, S-methylisourea, and 2-mercaptoethylguanidine. See also inhibitors disclosed in Hobbs et al., Annu Rev. Pharmacol. Toxicol. (1999), 39, pages 191-220; Salard et al., J Inorg Biochem 100, 2024-33 (December 2006) and http://www.caymanchem.com/app/template/scientificIllustrations%2CIllustration.vm/illustration/2056/image/preview/a/z;jsessionid=16F811460A0E2CD623A71C1614E69A2C. iNOS-specific inhibitors are preferred.

Specific non-limiting examples of useful NO scavengers include non-heme iron-containing peptides or proteins, porphyrins, metalloporphyrins, dithiocarbamates, dimercaptosuccinic acid, phenanthroline, desferrioxamine, pyridoxal isonicotinoyl hydrazone (PIH), 1,2-dimethyl-3hydroxypyrid-4-one (L1), [+] 1,2-bis (3,5-dioxopiperazine-1yl)propane (ICRF-187), 2-(4-carboxyphenyl)-4,5-dihydro-4,4,5,5-tetramethyl-1H-imidazolyl-1-oxy-3-oxide (Carboxy-PTIO), and the like. A preferred example of useful NO scavenger is a perfluorocarbon emulsion as disclosed in Rafikova et al., Circulation. 2004 Dec. 7; 110(23):3573-80.

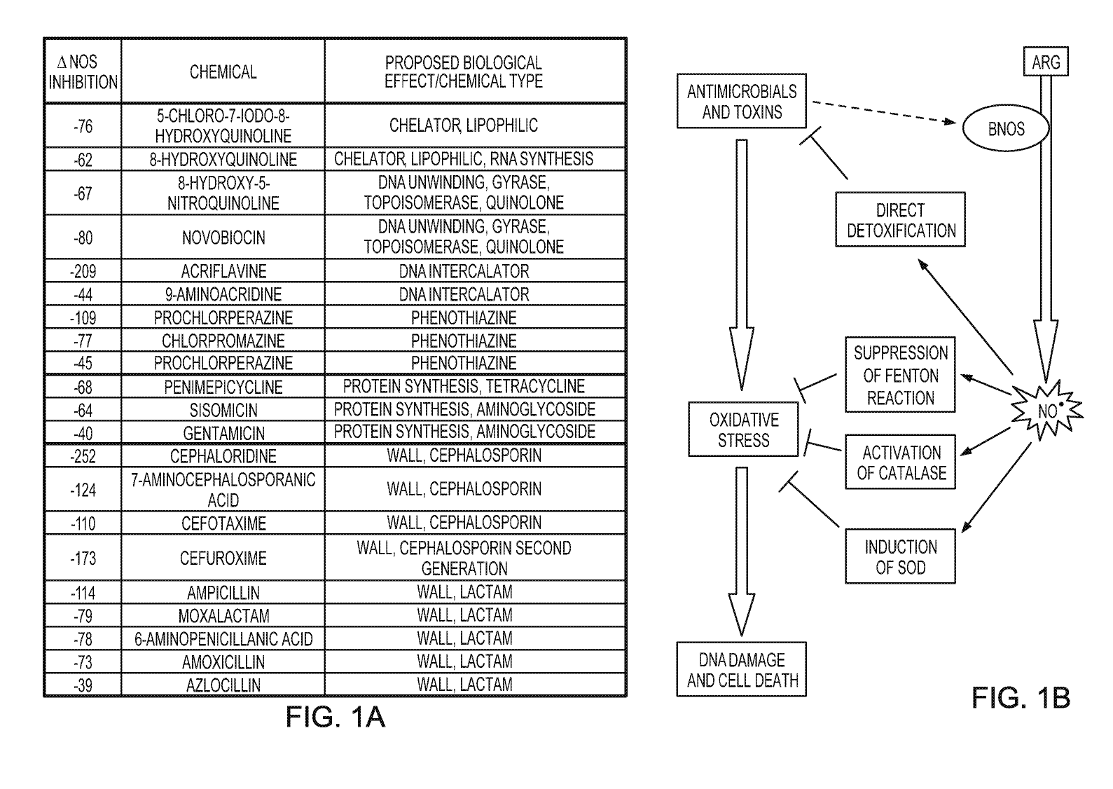

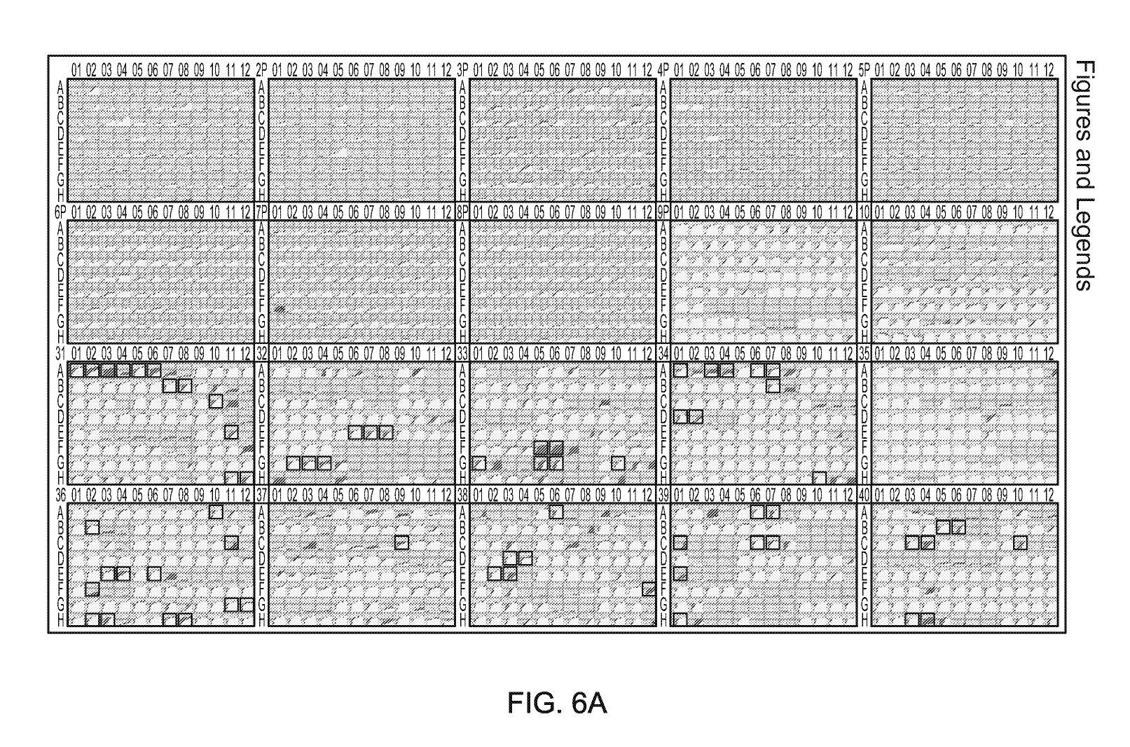

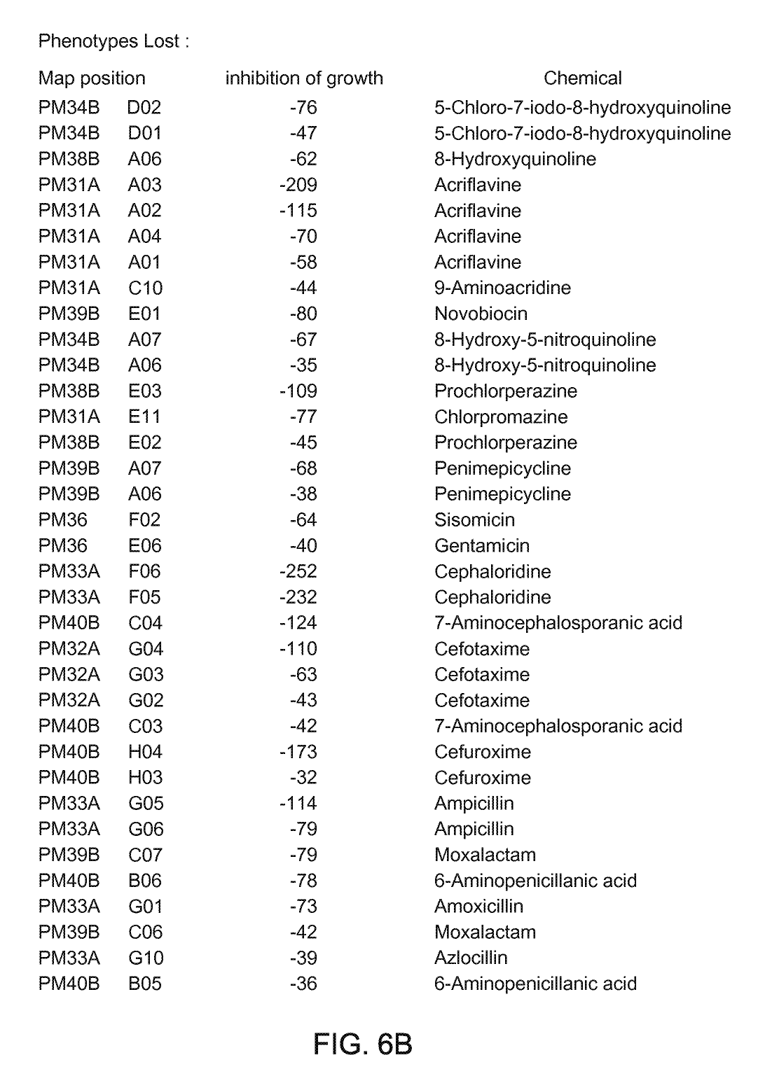

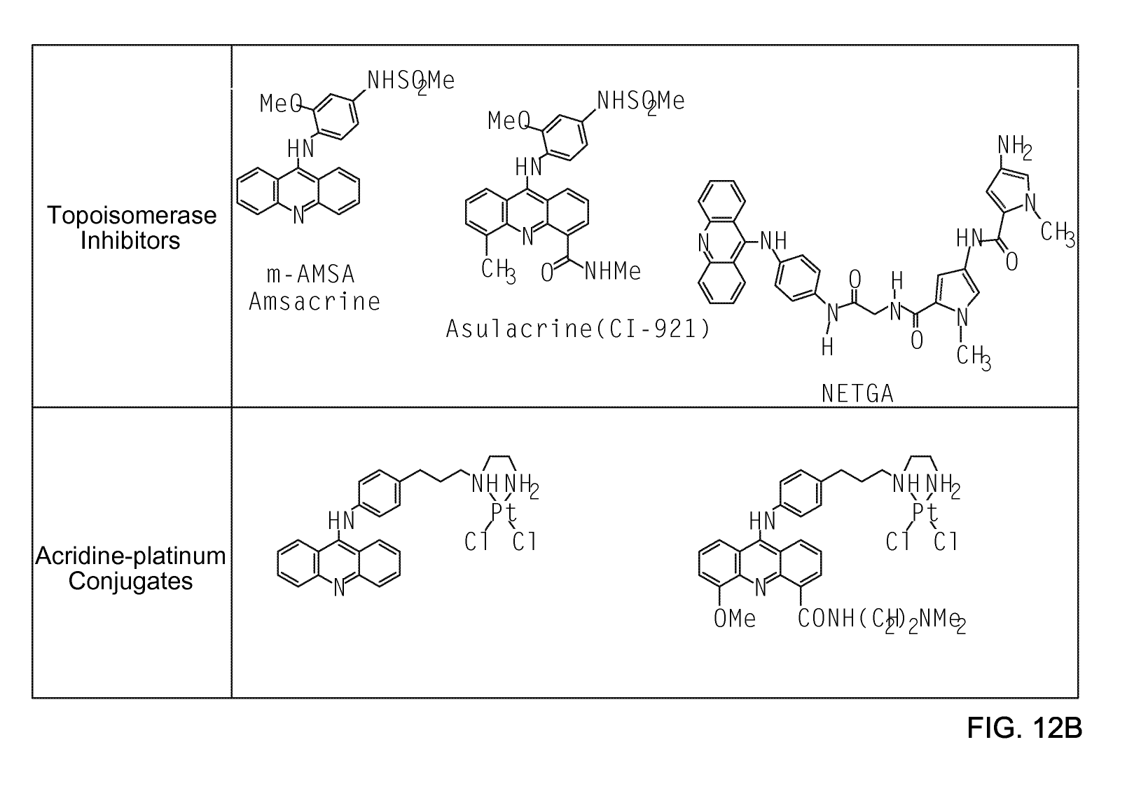

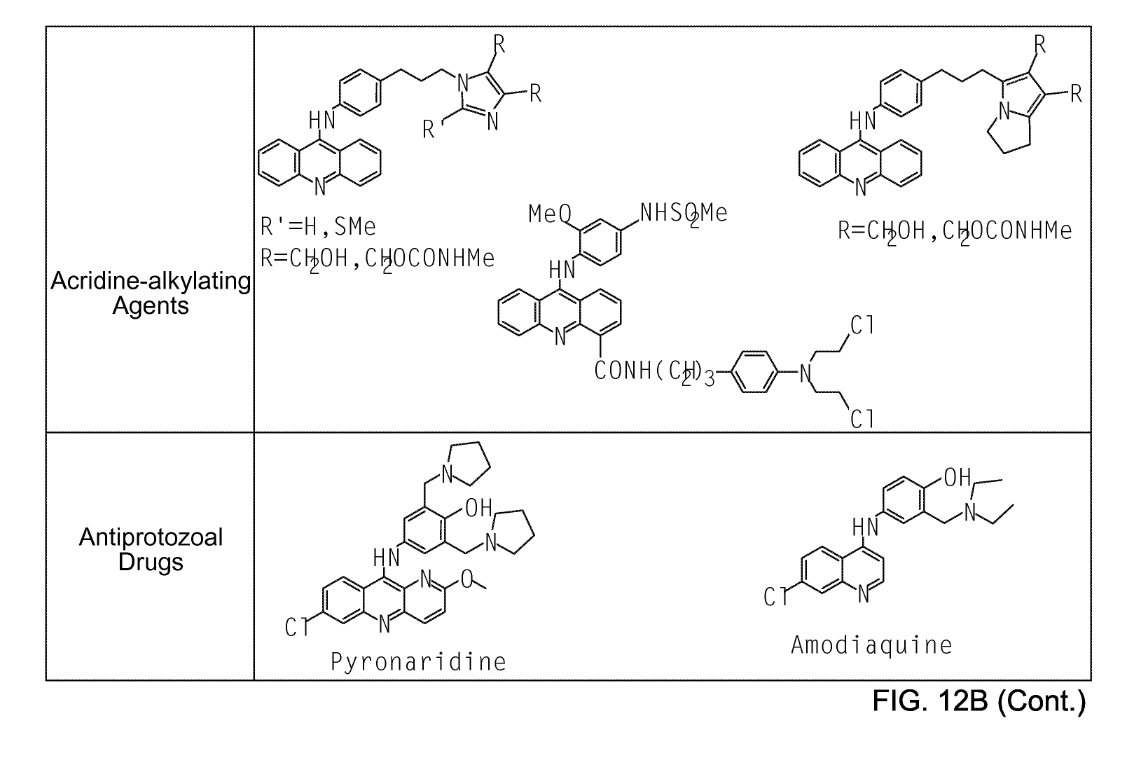

Specific non-limiting examples of antimicrobial compounds which become inactivated by NO and/or natural products of NO oxidation in vivo and/or become less effective due to NO action and therefore would benefit from the combination therapy of the invention are provided in FIGS. 1A and 6A-B and in Table 1, below. Specific non-limiting examples of anti-cancer and anti-protozoa compounds which become inactivated by NO and/or natural products of NO oxidation in vivo and/or become less effective due to NO action and therefore would benefit from the combination therapy of the invention are provided in FIG. 12B and Tables 2-4, below.

TABLE 1
Aminoacridine derivatives used as antimicrobials

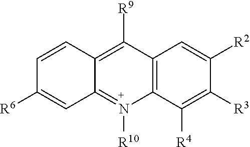

| | R² | R³ | R⁴ | R⁶ | R⁹ | R¹⁰ | MIC | Ref. |
|---|---|---|---|---|---|---|---|---|
| Proflavine | H | NH₂ | H | NH₂ | H | H | | 1 |
| Euflavine (Acriflavine, Panflavine, | H | NH₂ | H | NH₂ | H | CH₃ | 120 μM (19 μg/ml) S. aureus | 2 |
| Planacrine, Gonoflavin, Trypaflavin) | | | | | | | | |
| Diflavine | NH₂ | H | H | NH₂ | H | H | | 3 |
| Flavicid | CH₃ | NH₂ | H | (CH₃)₂N | H | CH₃ | 10 μM (~2.6 μg/ml) | 4 |
| | | | | | | | S. aureus | |
| Ethacridine (Rivanol) | CH₃CH₂O | H | H | NH₂ | NH₂ | H | | 5 |
| Aminacrine | H | H | H | H | NH₂ | H | | 6 |
| 3-amino-10-methyl-6-haloacridinium | H | NH₂ | H | Halogen | H | CH₃ | | 7 |
| 3-nitro-9-aminoacridine | H | NO₂ | H | H | NH₂ | H | 1.5 μM (~0.4 μg/ml) | 8 |
| | | | | | | | Streptococcus | |
| 9-Amino-2,3-dimethoxy-6-nitroacridine | CH₃O | CH₃O | H | NO₂ | NH₂ | O | 20 nM (~6 ng/ml) | 9 |
| 10-oxides | | | | | | | Streptococcus C203 | |
| Salacrin | H | H | CH₃ | H | NH₂ | H | | 10 |

Amino groups in positions 3, 6 and 9 considered to be crucial for bacteriotoxicity. These and other acridine derivatives which have primary arylamino groups can be efficiently deaminated by NO⁺.
1. Rank, B. K. (1944). Use and abuse of local antiseptics on wounds. Medical Journal of Australia 31, 629-36.
2. Browning, C. H. (1943). The present status of aminoacridine compounds (flavines) as surface antiseptics. British Medical Journal i, 341-3.
3. Turnbull, H. (1944). A rational treatment of gunshot wounds of long bones with established sepsis. Australian and New Zealand Journal of Surgery 14, 3-13.
4. Langer, H. (1920). Zur theorie der chemotherapeutischen leistung. Nach versuchen an akridinium-farbstoffen. Deutsche Medezinische Wochenschrift 46, 1015-6.
5. Levrat, M. & Morelon, F. (1933). Contribution à l'étude pharmacodynamique et toxicologique de la trypaflavine, du rivanol et d'autres dérivés de l'acridine. Bulletin Science Pharmacologique 40, 582-92.
6. Poate, H. G. (1944). Acridines in septic wounds. Use of 5-aminoacridine. Lancet ii, 238-40.
7. British Patent no. 367,037-I. G. Farben, Process for the manufacture of acridine derivatives (1932).
8. Albert, A., Rubbo, S. D., Goldacre, R. J., Davey, M. E. & Stone, J. D. (1945). The influence of chemical constitution on antibacterial activity. Part II: a general survey of the acridine series. British Journal of Experimental Pathology 26, 160-92.

TABLE 2
Anticancer acridine derivatives which can be inactivated by NO°

| | |
|---|---|
| Topoisomerase inhibitors | 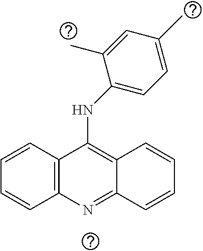 |
| | 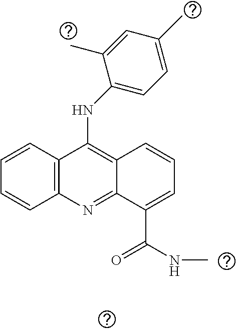 |
| | 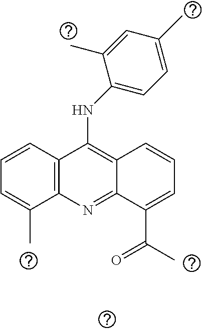 |
| | 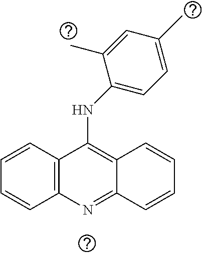 |
| | 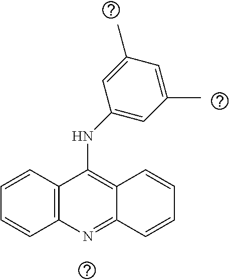 |
| | 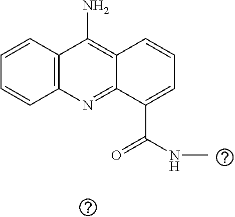 |
| | 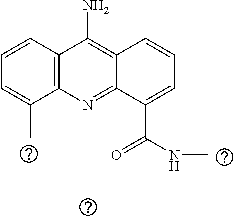 |
| | 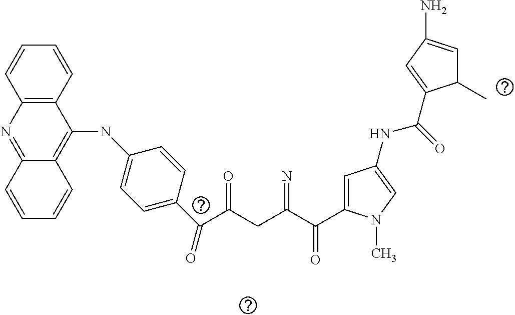 |
| Acridine-platinum conjugates | 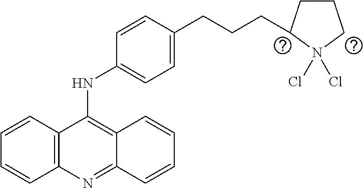 |
| | 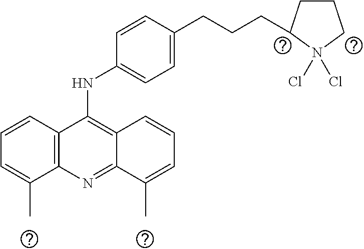 |
| Acridine-alkylating agents | 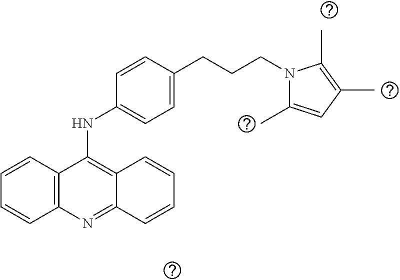 |
| | 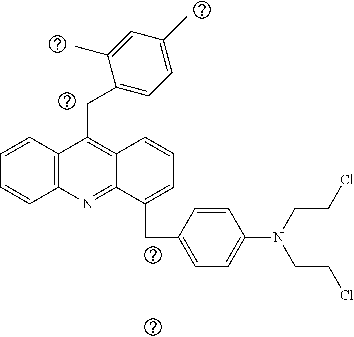 |
| | 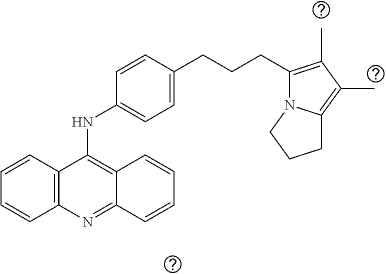 |
| Telomerase inhibitor | 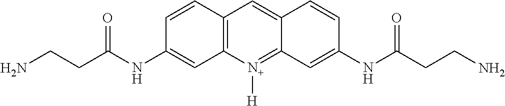 |
| DNA crosslinking | 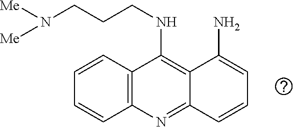 |

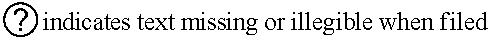

TABLE 3
Examples of anti-cancer drugs that can be inactivated by NO

| Type | Class | Examples |
|---|---|---|
| Antitumor Antibiotics | Anthracyclines | Doxorubicin |
| | | Daunorubicin |
| | | Mitoxantrone |
| | Chromomycins | Actinomycin D |
| | | Mithramycin A |
| | Other | Mitomycin C |
| | | Bleomycin |
| Plant Alkaloids | Vinca alkaloids | Vincristine |
| | | Vinorelbine |
| | Taxanes | Paclitaxel |
| | | Docetaxel |
| | Camptothecan analogs | Irinotecan |
| | Topoisomerase I inhibitors | Topotecan |
| | | Fumitremorgin C |

TABLE 4
Examples of aminoacridine-based anti-protozoal drugs that can be inactivated by NO

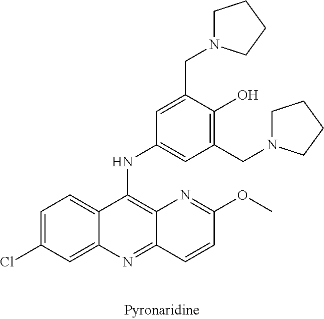

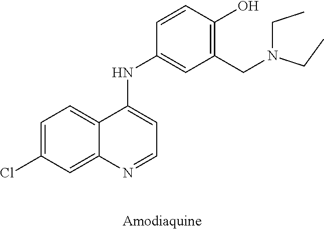

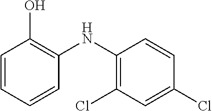

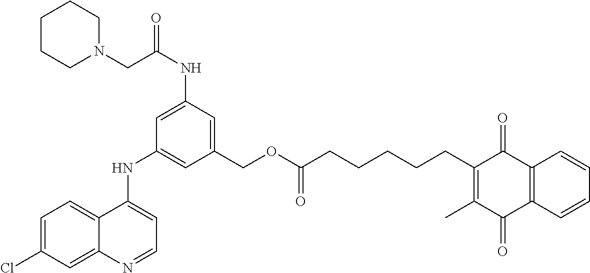

Specific non-limiting examples of microbial infections for which the method of the present invention would provide an advantageous treatment include S.aureus and B. anthracis infections. Other preferred examples include microbial infections causing pneumonia and endocarditis (e.g., S.aureus infection). Specific non-limiting examples of protozoal infections for which the method of the present invention would provide an advantageous treatment include malaria.

In a related aspect, the present invention provides a method for decreasing an effective concentration of a drug used in an antibacterial, anti-protozoa or chemotherapeutic treatment, wherein said drug becomes inactivated by NO and/or natural products of NO oxidation in vivo and/or becomes less effective due to NO action, said method comprising co-administering said drug with an inhibitor of endogenous NO production and/or NO scavenger. This method of the invention allows to diminish side-effects of potentially toxic antibacterial, anti-protozoa or chemotherapeutic treatments.

### Definitions

The phrases “reactive species of nitric oxide” or “reactive NO species” mean the chemicals capable of nitrosation and nitration of target macromolecules, e.g. N₂O₃, N₂O₄, ONOO—, and .NO₂. Peroxynitrite anion (ONOO⁻) and nitrogen dioxide (.NO₂), are formed as secondary products of .NO metabolism in the presence of oxidants including superoxide radicals (O_{2.}⁻), hydrogen peroxide (H₂O₂), and transition metal centers.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In the context of the present invention insofar as it relates to any of the disease conditions recited herein, the terms “treat”, “treatment”, and the like mean to relieve or alleviate at least one symptom associated with such condition, or to slow or reverse the progression of such condition.

The phrase “pharmaceutically acceptable”, as used in connection with compositions of the invention, refers to molecular entities and other ingredients of such compositions that are physiologically tolerable and do not typically produce untoward reactions when administered to an animal such as a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “administering” or “administration” are intended to encompass all means for directly and indirectly delivering a compound to its intended site of action. The compounds of the present invention can be administered locally to the affected site (e.g., by direct injection into the affected tissue) or systemically. The term “systemic” as used herein includes parenteral, topical, oral, spray inhalation, rectal, nasal, and buccal administration. Parenteral administration includes subcutaneous, intravenous, intramuscular, intra-articular, intra-synovial, intrasternal, intrathecal, intrahepatic, intralesional, and intracranial administration.

The terms “animal” and “subject” mean any animal, including mammals and, in particular, humans.

As used in this specification and the appended claims, the singular forms “a,” “an,” and “the” include plural references unless the context clearly dictates otherwise.

In accordance with the present invention, there may be employed conventional molecular biology, microbiology, and recombinant DNA techniques within the skill of the art. See, e.g., Sambrook, Fritsch and Maniatis, Molecular Cloning: A Laboratory Manual, 2nd ed., Cold Spring Harbor Laboratory Press, Cold Spring Harbor, N.Y., 1989 (herein “Sambrook et al., 1989”); DNA Cloning: A Practical Approach, Volumes I and II (Glover ed. 1985); Oligonucleotide Synthesis (Gait ed. 1984); Nucleic Acid Hybridization (Hames and Higgins eds. 1985); Transcription And Translation (Hames and Higgins eds. 1984); Animal Cell Culture (Freshney ed. 1986); Immobilized Cells And Enzymes (IRL Press, 1986); B. Perbal, A Practical Guide To Molecular Cloning (1984); Ausubel et al. eds., Current Protocols in Molecular Biology, John Wiley and Sons, Inc. 1994; among others.

### EXAMPLES

The present invention will be better understood by reference to the following non-limiting examples.

### Example 1

### Endogenous Nitric Oxide Protects Bacteria Against a Wide Spectrum of Antibiotics

### Methods and Materials

### Strains and Growth Conditions

B.subtilis, S.aureus and P.aeruginosa strains were grown in Luria-Bertani (LB) broth or on LB plates supplemented with 1.5% Bacto agar at 37° C. Construction of nos deletion and baNOS overexpression strains in domesticated B.subtilis 168 (trpC2) background were described in previous publications (1, 2). nos deletion in undomesticated B.subtilis 6051 (NCIB 3610) strain was produced according to Kobayashi K. method (3). Briefly, the genomic DNA from B.subtilis Δnos (his leu met nos::Spc) strain (1) was transformed into B.subtilis 6051 and the spectinomycin resistant prototrophic colonies were selected on minimal media. B.anthracis strains were grown in BHI media supplemented with glycerol at 37° C. nos deletion in B.anthracis Sterne strain was described previously (4). S.aureus nos deletion mutant was generated according to (10).

Some growth curves were obtained on Bioscreen C automated growth analysis system. For these experiments overnight cultures of bacteria were diluted first saline till OD600=0.1 for S.aureus and OD600=0.25 for B.subtilis. These stocks were used as 10× to inoculate 100-wells microplates filled with LB or LB +corresponding antimicrobial. Plates were incubated in the Bioscreen C with maximum shaking at 37° C. OD600 determined every 30 min and the means of triplicates plotted.

P.aeruginosa PA-14 strain was from Ausubel F., PA-01 and ΔphzA1 strains were University of Washington Pseudomonas aeruginosa mutant library. Coculture experiments were preformed according to Farrow J., et at (5). To stimulate PYO synthesis by P.aeruginosa Pseudomonas agar P was used as a solid media. Plates were incubated at 37° C. and the diameters of lysis zones measured three to seven days latter.

### Chemicals and Regents

All chemicals were from Sigma, except PYO which was purchased from Cayman.

### Mammalian Tissue Culture

The human hepatoblastoma cell line HepG2 (American Type Culture Collection, Manassas, Va., USA) was grown at 37° C. with 5% CO2 in Dulbecco's Modified Eagle's Medium (Gibco BRL, Grand Island, N.Y.), supplemented with 10% fetal calf serum, 2 mM L-glutamine, and 50 mg/ml 2 gentamycin. Cells were inoculated in 24-well plates and grown till ˜50% of confluence. 20 μM AMSAcrine, 100 μM MAHMA NONOate, or their mixture was added to the triplets of wells. Control wells were left untreated. To inhibit iNOS expression, 4 mM L-NAME or 100 μM L-NIL was added to indicated wells for 8 hours prior to AMSAcrine addition. Cell viability was estimated 24 hours later by the CellTiter 96® Aqueous Cell Proliferation Assay (Promega). To monitor AMSAcrine degradation Dulbecco's Modified Eagle's Medium (Gibco BRL, Grand Island, N.Y.) without phenol red and gentamycin was used. Cells were inoculated in 24-well plates and grown till ˜80% of confluence. 15 μM AMSAcrine was added to the quadruplets of wells. Control wells had AMSAcrine but no cells in them. To inhibit iNOS expression, 4 mM L-NAME or 100 μM LNIL was added for 6 hours prior to AMSAcrine addition.

Introduction

Bacterial NO-synthases (bNOS) are present in many Gram-positive species and have been demonstrated to synthesize NO from arginine in vitro and in vivo. However, the physiological role of bNOS remains largely unknown. It is shown herein that NO generated by bNOS increases the resistance of bacteria to a broad spectrum of antibiotics, enabling them to survive and share habitats with antibiotic-producing microorganisms. NO-mediated resistance is achieved through both chemical modification of toxic compounds and alleviation of the oxidative stress imposed by many antibiotics. It is further shown herein that NO-mediated detoxification occurs in mammalian cells as well. Therefore, suppressing mammalian and bacterial NOS activities should be considered as a means to enhance the effectiveness of standard chemo- and antimicrobial therapies.

bNOS and its eukaryotic counterparts, which produce NO by catalyzing the oxidation of L-arginine to L-citrulline, are structurally and mechanistically related (1-3). Although bNOS lacks the essential reductase domain, it uses available cellular reductases to generate NO in vivo (4). Previously, it has been demonstrated that bNOS protects bacteria against oxidative stress (5, 6). This function of bNOS was found to be essential for some pathogenic organisms. For example, the survival of Bacillus anthracis (B.anthracis) in macrophages strictly depends on bNOS activity, which is an important virulence factor that protects this pathogen from immunological oxidative bursts (6). bNOS has also been shown to function during Streptomyces turgidiscabies infection of plants (7). bNOS genes are also present in the genomes of numerous nonpathogenic soil bacteria (4) (Table 5), arguing for the existence of hitherto unknown selective pressures imposed by their natural habitats that favor endogenous NO production.

TABLE 5
A representative list of bacteria that possess eukaryotic-like NOS.

| | Microorganism | Phyla | Pathogenicity |
|---|---|---|---|
| 1 | Bacillus subtilis | Firmicutes | Non pathogenic |
| 2 | Bacillus amyloliquefaciens | | |
| 3 | Bacillus licheniformis | | |
| 4 | Bacillus pumilus | | |
| 5 | Geobacillus kaustophilus | | |
| 6 | Bacillus halodurans | | |
| 7 | Bacillus clausii | | |
| 8 | Oceanobacillus iheyensis | | |
| 9 | Exiguobacterium sibiricum | | |
| 10 | Geobacillus stearothermophilus | | |
| 11 | Deinococcus geothermalis | Deinococcus- | Non pathogenic |
| 12 | Deinococcus radiodurans | Thermus | |
| 13 | Rodococcus sp. | Actinobacteria | Non pathogenic |
| 14 | Streptomyces avermitilis | | |
| 15 | Salinispora arenicola | | |
| 16 | Saccharopolyspora erythraea | | |
| 17 | Streptomyces turgidiscabies | Actinobacteria | Plant Pathogenic |
| 18 | Streptomyces scabiei | | |
| 19 | Bacillus anthracis | Firmicutes | Pathogenic |
| 20 | Bacillus cereus | | |
| 21 | Bacillus thuringiensis | | |
| 22 | Bacillus weihenstephanensis | | |
| 23 | Staphylococcus aureus | | |
| 24 | Staphylococcus haemolyticus | | |
| 25 | Staphylococcus epidermidis | | |
| 26 | Staphylococcus saprophyticus | | |
| 27 | Natronomonas pharaonis | Archaea | Non pathogenic |

bNOS Protects Bacteria Against a Broad Range of Antibiotics

To elucidate the physiological role of bNOS, wild type (wt) and nos deficient Bacillus subtilis (B.subtilis) strains were compared in the Phenotype MicroArray from Biolog, Inc (FIG. 6A). PM technology allows the monitoring of microorganisms under multiple growth and stress conditions and, thereby, the simultaneous profiling of hundreds of phenotypes. Remarkably, whereas wt and nos mutant strains showed no growth differences in various media and nutrient supplements, a large number of bactericidal chemicals preferentially suppressed growth of the nos mutant (FIG. 1A and FIG. 6A-B). Despite their highly variable structure, these chemicals could be arranged in three major groups. The first group consists of quinolones, acridines and phenothiasines. They contain condensed aromatic rings that share a planar structure capable of DNA intercalation and bacterial killing by inhibition of topoisomerase and/or gyrase (8-10). The second group includes protein synthesis inhibitors, and the third group includes lactams that inhibit cell wall biosynthesis. Although these three groups of chemicals are structurally unrelated, the striking ability of NO to compromise their bactericidal effects suggests that they manifest their antibacterial activity via a common mechanism that is targeted by endogenous NO. Indeed, it has been shown recently that bactericidal antibiotics, such as lactams, aminoglycosides, and quinolones, exert their toxicity, at least in part, by promoting reactive oxygen species (ROS) formation (9, 11, 12). On the contrary, NO protects Gram-positive bacteria against oxidative stress by a mechanism, which, in principle, could also explain NO-mediated cell resistance to antibiotics (5, 6) (FIG. 1B). To test this hypothesis and to determine the mechanisms of NO-mediated antibiotic resistance the inventors performed detailed analyses of the effects of NO on bacterial killing by three different, representative antimicrobials, acriflavin, pyocianine, and cefuroxime.

### The Dual Mechanism of NO-Mediated Protection Against Acridine-Type Antibiotics.

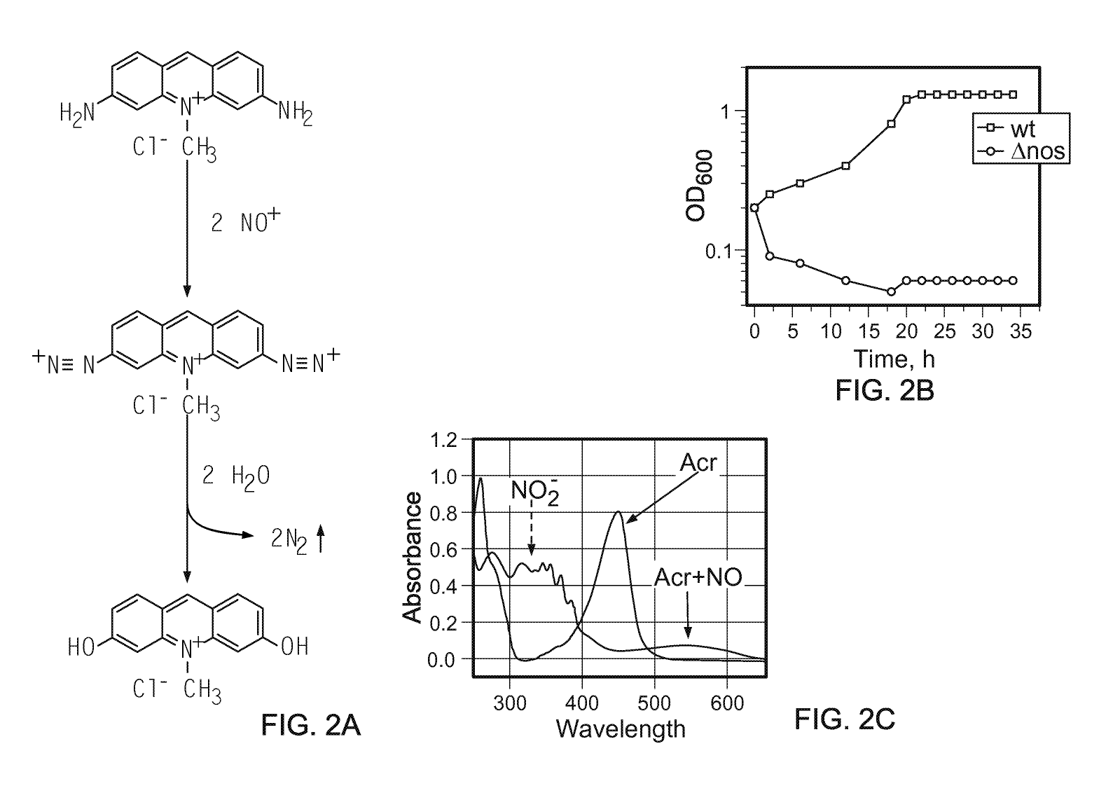

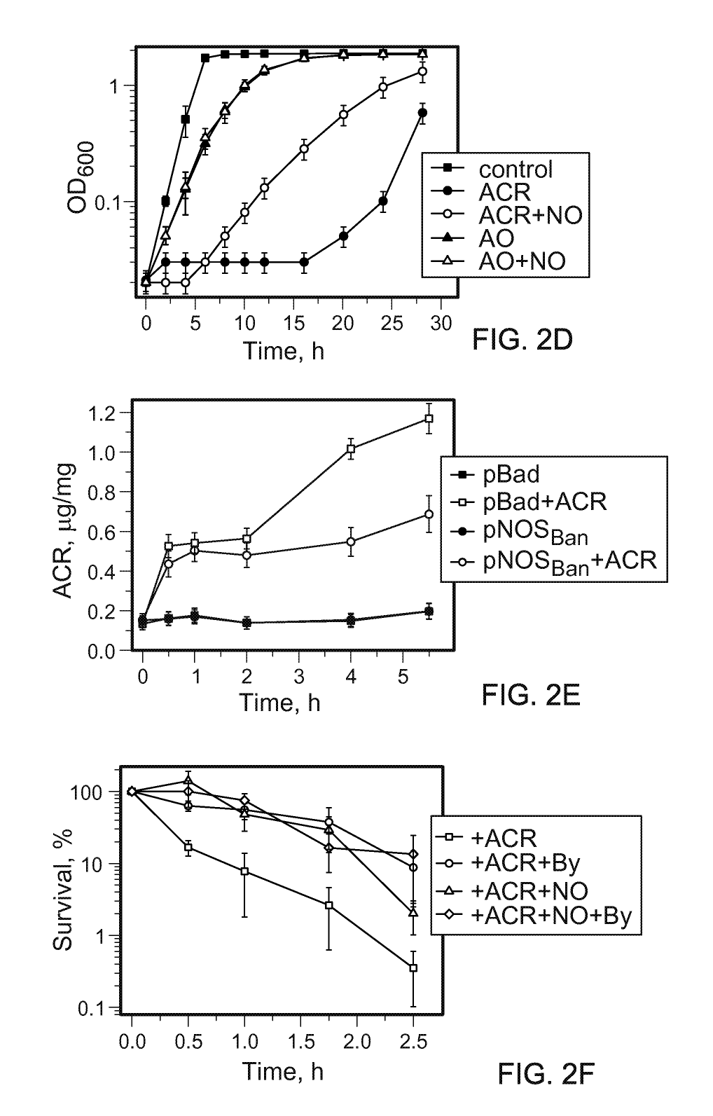

Acriflavine (ACR; FIG. 2A) is the most potent group A compound inhibitor of the Δnos strain (FIGS. 1A, 2B and 7A). It is an acridine-type antimicrobial with one of the highest potencies of the members of its class (e.g. MBC≦19 μM for S.aureus) (13). ACR carries two aromatic amino groups that are essential for its toxicity (10) (FIG. 2A). Products of NO oxidation (NO⁺) readily nitrosate arylamino moieties (FIG. 2A) (14). NO does not react with nucleophiles directly. However, products of NO oxidation (NO⁺) readily nitrosate arylamino moieties (FIG. 2A). Such products appear intracellularly via reaction with transition metals (such as Fe3+or Cu2+). Also, NO auto-oxidation is accelerated dramatically due to the process of micellar catalysis, which is mediated by proteins hydrophobic pockets and membranes in vivo. The resulting aryldiazonium cations are quickly hydrolyzed with the release of N₂ gas and the formation of less toxic dihydroxyacridine derivatives (FIG. 2A). Indeed, mixing ACR with NO resulted in gas formation and a color change from orange to faint blue. Spectral data support the proposed reaction (FIG. 2C): the 450 nm peak of ACR is converted into a wider ˜550 nm peak resulting from the byproducts of intermolecular diazonium crosslinks (14). To demonstrate that NO detoxifies ACR directly, ACR with NO were premixed in approximately equimolar amounts in growth media prior to inoculating it with bacteria. This resulted in reduced killing of either B.subtilis or S.aureus by the ACR (FIGS. 2D and 7). NO by itself did not affect the growth of bacteria at this concentration. Moreover, acridine orange (AO), in which the arylamino groups are methylated and unable to interact with NO⁺, was unaffected by NO treatment (FIG. 2D). The antibacterial effect of AO was significantly less than that of ACR, apparently due to the methylated NH₂ groups. These results indicate that bNOS-generated NO modifies ACR directly, thereby decreasing its potency in vivo.

To demonstrate that endogenous NO caused ACR modification, an E.coli strain expressing B.anthracis NOS was utilized. This strain produces NO upon induction with arabinose (4). ACR accumulation can be monitored directly in vivo owing to changes in its characteristic yellow color. As shown in FIG. 2E, the rate of intracellular ACR accumulation and its overall concentration diminished greatly in NO producing cells as compared to the empty vector control. We, therefore, conclude that the products of endogenous NO oxidation reacted with amino groups of ACR.

The direct reaction of ACR with NO⁺ reduced its toxicity only partially (FIG. 2D), suggesting that the efficient protection against ACR by endogenous NO involves additional mechanism(s). Since quinolones kill E.coli by promoting oxidative stress (11), and NO protects cells against ROS (4-6), it was examined whether ACR also kills bacteria via ROS formation. Pretreatment of cells with bipyridyl, an iron chelator that efficiently suppresses the damaging Fenton reaction (15), substantially decreased the toxicity of ACR (FIG. 2F). Furthermore, NO pretreatment (3 min prior to antibiotic addition) was as effective as bipyridyl in protecting against ACR (FIG. 2F), but failed to further protect cells previously pretreated with bipyridyl (FIG. 2F), indicating that both chemicals acted through the same pathway, i.e. by suppressing the Fenton reaction. A direct interaction between NO and antibiotics was excluded because NO has a very short life in biological solutions and must have been eliminated as nitrite within the first seconds of pretreatment (16, 17). Together, the ACR results lead to two conclusions: (i) ACR kills bacteria, at least in part, by a ROS-dependent mechanism; (ii) The mechanism of NO-mediated protection against ACR is two-fold; NO directly modifies ACR, making it less toxic, and, at the same time, it also protects against ACR-induced oxidative stress.

bNOS Contributes to Bacilli Fitness and Resistance to Natural Toxins.

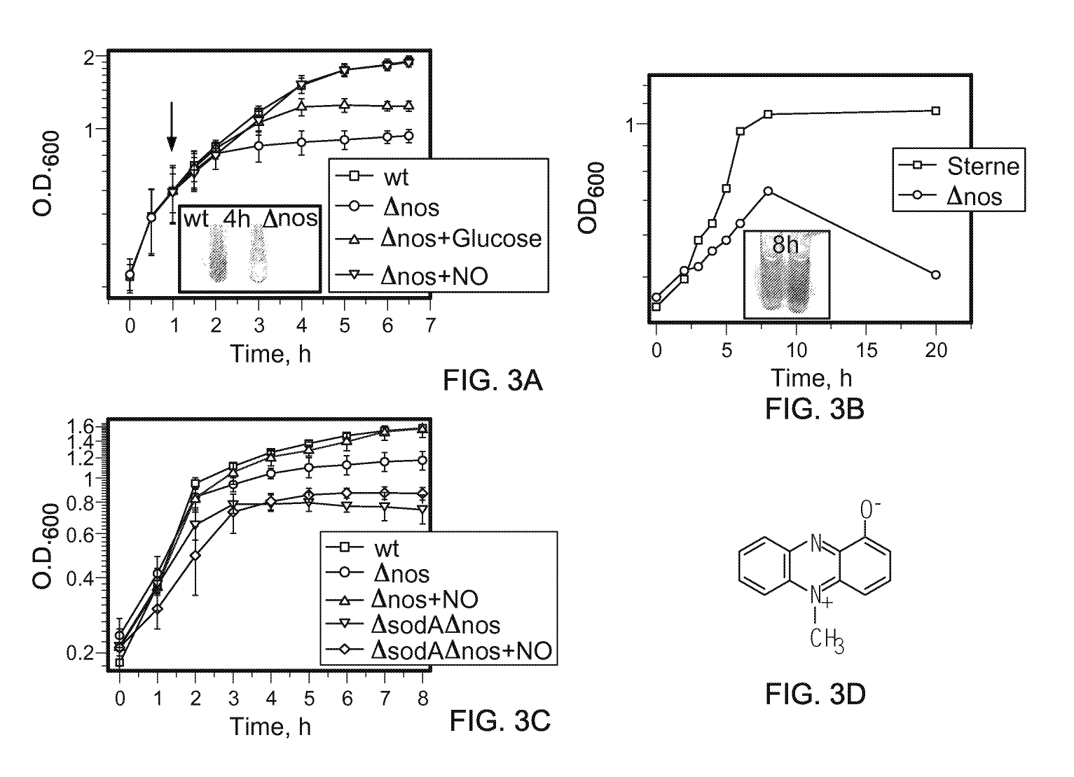

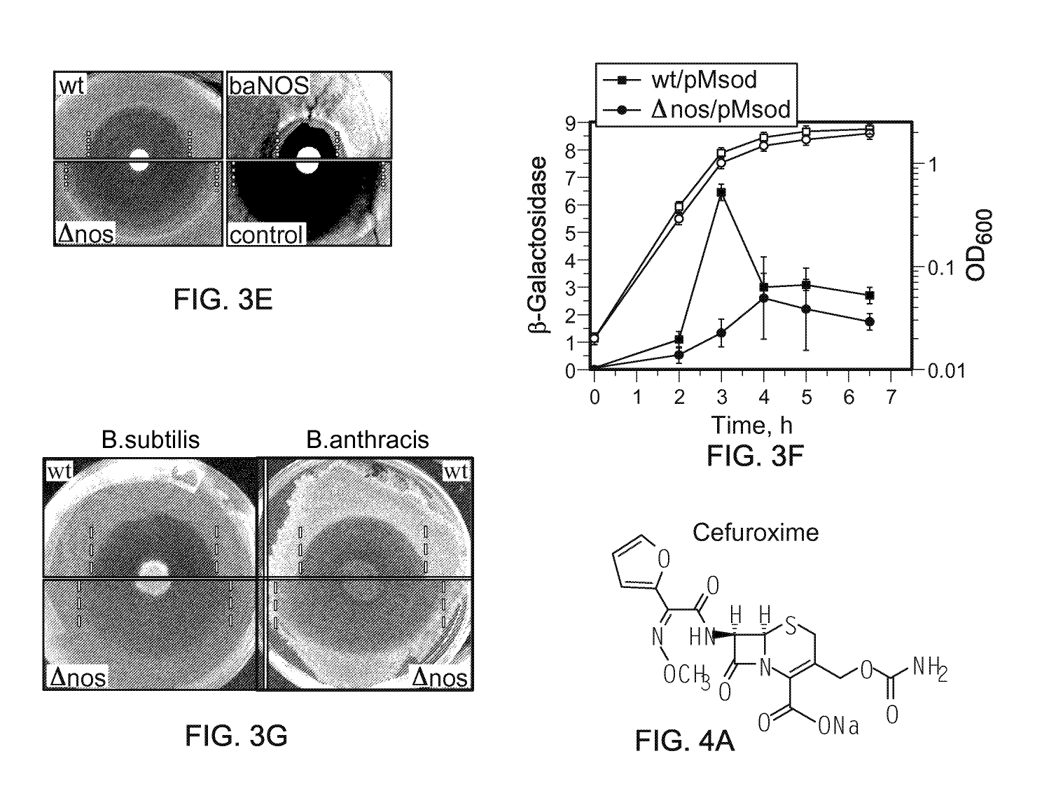

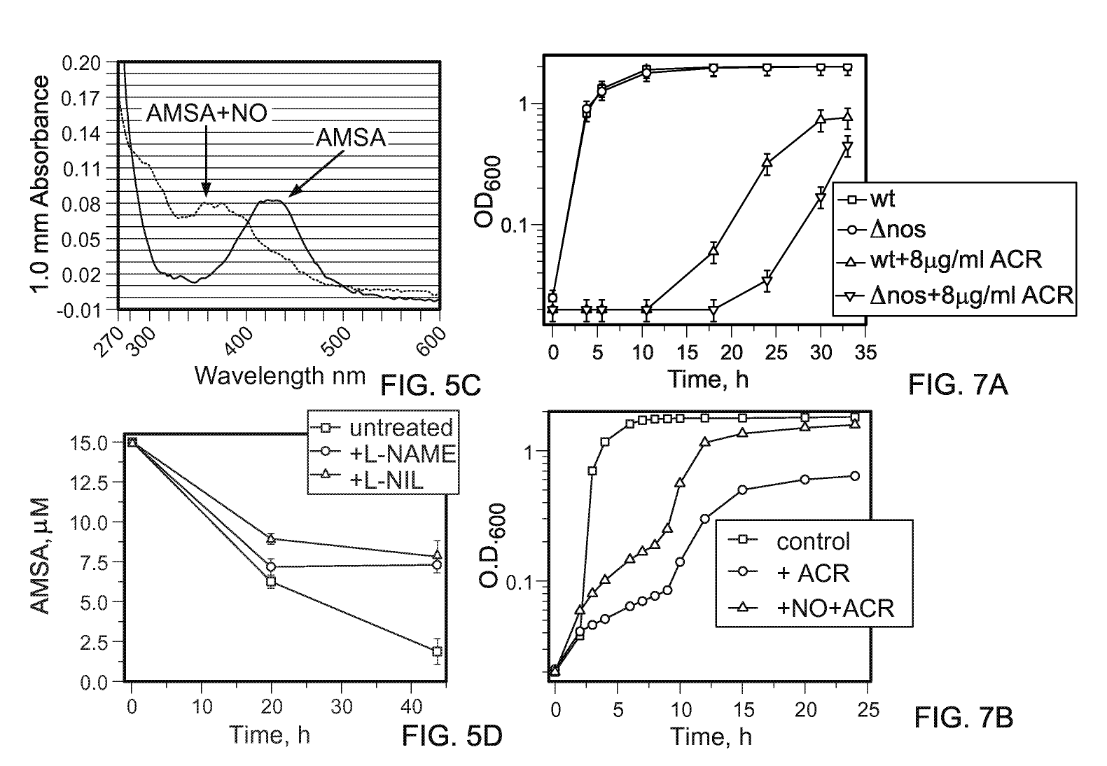

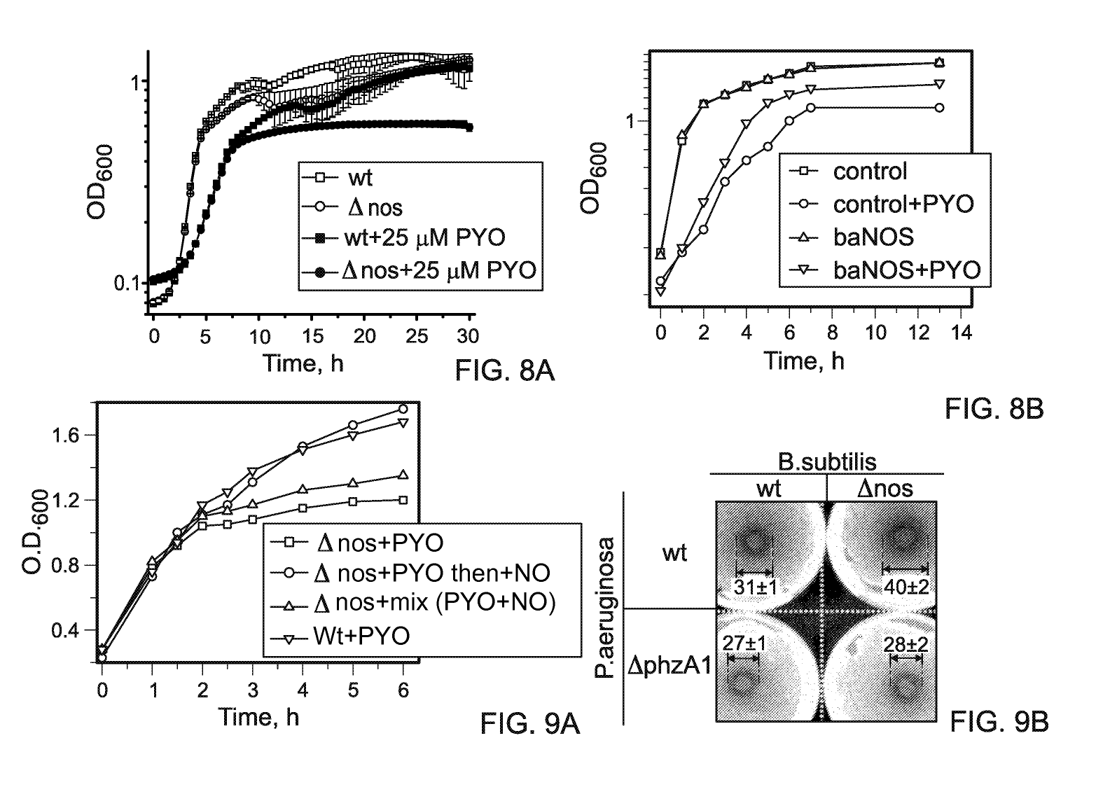

Pyocyanin (1-hydroxy-5-methyl-phenazine, PYO) is one of many antimicrobials that resemble ACR structurally (FIG. 3D). It is a natural toxin synthesized by Pseudomonas aeruginosa (P.aeruginosa) and has broad clinical effects. During P.aeruginosa infection, PYO inhibits mammalian cell respiration, disrupts ciliary movement, and suppresses epidermal cell growth and lymphocyte proliferation (18-20). P.aeruginosa virulence depends on PYO (21) and correlates with its concentration in the pulmonary secretions of cystic fibrosis patients (22). PYO is also a potent antibiotic against a wide variety of microorganisms (23, 24). Since both P.aeruginosa and B.subtilis inhabit the same soil niche, the inventors hypothesized that endogenous NO could defend B.subtilis against PYO. Indeed, PYO inhibited the growth of the nos deletion strain to a much greater extent than it did of wt B.subtilis (FIG. 3A). In contrast to the Δnos cells, which ceased growth in liquid culture within 3 hours of incubation with PYO, the wt cells continued to grow and metabolized all the PYO, as evidenced by the disappearance of its characteristic blue color (FIG. 3A, insert [the intensity of grey corresponds to bacterial growth]). A similar result was obtained with cells grown on agar plates (FIG. 3E, left): the PYO killing zone was significantly smaller for the wt cells than for the Δnos cells. Moreover, addition of exogenous NO completely restored the growth of the Δnos cells in the presence of PYO (FIG. 3A). Such NO-mediated growth recovery occurs even though NO was added hours prior to the onset of growth inhibition by PYO (FIG. 3A), arguing that NO signaling initiates a mechanism of persistent defense against PYO. Finally, to unambiguously demonstrate the protective effect of endogenous NO against PYO, the B.anthracis nos gene was integrated into the chromosome of B.subtilis Δnos cells under control of the arabinose inducible promoter (4). Expression of B. anthracis NOS (baNOS) increased the resistance to PYO both in liquid culture and on agar plates (FIGS. 3E, right and 8B). Consistently, deletion of the nos gene in B.anthracis dramatically sensitized them to PYO (FIG. 3B). This sensitization was stronger than the sensitization resulting from nos gene deletion in B.subtilis, and correlates with the greater intrinsic activity of baNOS than of bsNOS (4, 6). Because deletion of bNOS in S.aureus also sensitized this pathogen to PYO (FIG. 8A), these results, taken together, demonstrate that NOS-mediated protection against PYO is a general phenomenon of all NOS-containing bacteria.

NO-mediated protection against PYO would render Bacilli more resistant to P.aeruginosa during competition for nutrients in soil. To recapitulate this natural situation P.aeruginosa was co-cultured with B.subtilis and B.anthracis on P agar, which stimulates PYO production. A drop of P.aeruginosa PA14 was placed atop a Bacilli lawn for overnight incubation (FIG. 3G). PA14 is a clinical isolate that produces a high level of PYO (25, 26). PA14 kills both B.subtilis and B.anthracis. However the lysis zones were significantly larger for the nos mutant cells than for the wild type cells of both species (FIG. 3G). To verify that NOS-dependent cell viability was indeed due to PYO detoxification by NO in vivo, the inventors utilized P.aeruginosa deficient in PYO production, PYO(−). The PYO(−) mutant made smaller lysis zones of equal size for both Δnos and wt B.subtilis (FIG. 9B). We, therefore, concluded that PYO is one of the key factors that P.aeruginosa uses to combat Bacilli. Bacilli, however, utilize endogenous NO to reduce the oxidative stress associated with PYO toxicity (see below), thereby defending themselves against killing by P.aeruginosa.

bNOS-Dependent Activation of Superoxide Dismutase is Required for Pyocyanin Protection.

In contrast to ACR, PYO does not have arylamino groups to react with NO⁺. Consistently, premixing NO with PYO did not result in a color change or attenuation of PYO toxicity (FIG. 9A), (27), arguing that NO-mediated protection was not due to direct chemical interaction between NO⁻ and PYO, but, rather, required a cellular response mechanism. The inventors noticed that PYO did not significantly affect the exponential phase of growth of either the wt or the Δnos cells, but did arrest the growth of the Δnos cells at the stationary growth phase (FIG. 3A). B.subtilis catabolizes glucose and other sugars to pyruvate during exponential growth. Instead of oxidizing pyruvate further they excrete it as acetoin, thereby limiting the respiratory chain activity. In contrast, in the stationary phase, acetoin is reused from the media leading to the increase of oxidative phosphorylation. During the stationary phase, when bacteria are fully engaged in respiration, a redox cycling agent, such as PYO, can strip electrons from the semi-reduced menaquinone (an intermediate of the electron transport chain) and donate them to free oxygen, thereby promoting superoxide anion formation. Indeed, PYO toxicity has been associated with ROS (23, 24). Consistently, the presence of glucose, which prolonged fermentative growth, delayed the onset of PYO growth inhibition (FIG. 3A).

The role of superoxide dismutase (SOD) was investigated to confirm that PYO toxicity is indeed associated with ROS. B.subtilis carries only one SOD (SodA), which confers resistance to endogenous superoxide and superoxide generating agents (28, 29). Deletion of sodA rendered B.subtilis highly sensitive to PYO (FIG. 3C), which validates the relationship between PYO and superoxide production in vivo. Moreover, exogenous NO did not protect the sodA-deficient strain against PYO (FIG. 3C), suggesting that NO functions in the control of sodA expression. To investigate the relationship between NO and SOD further, the expression of sodA in wt and Δnos strains was compared (FIG. 3F). SodA expression increased sharply in wt cells at the late exponential phase of growth (FIG. 3F) (28). This distinctive spike of SodA expression was abolished in Δnos cells (FIG. 3F). Taken together, these results indicate that bNOS is required for SodA activation, which, in turn, provides resistance to PYO. The intriguing mechanism of bNOS regulation of SodA, which is likely to involve NO/NO⁺ signaling, is a subject of ongoing investigation.

bNOS Activation is a General Defense Response Against Antibiotics.

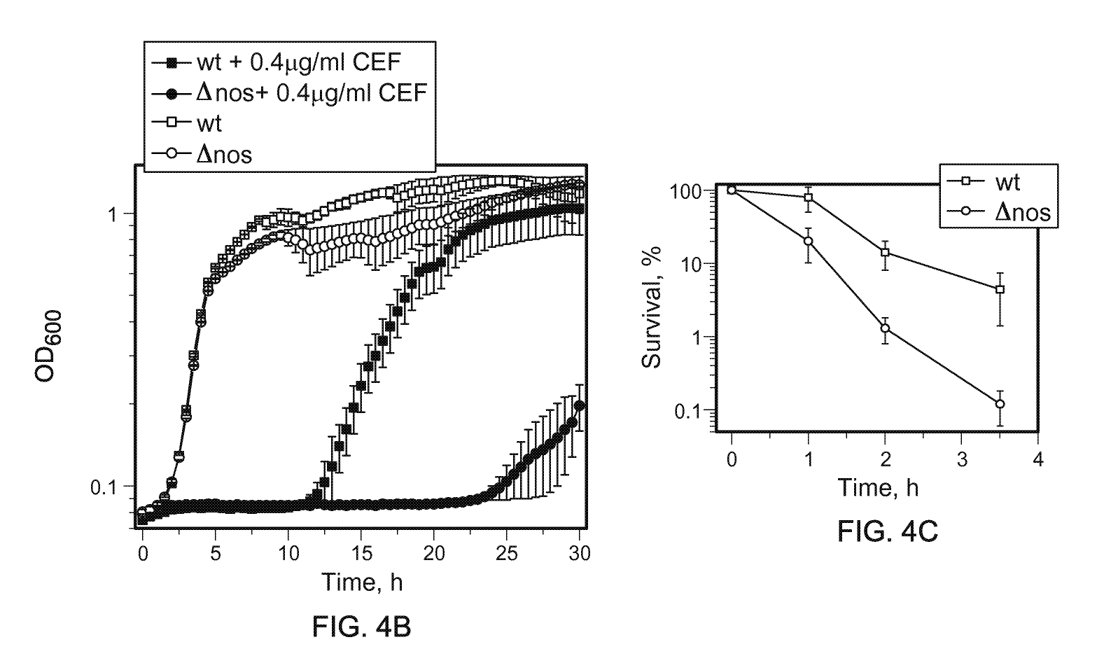

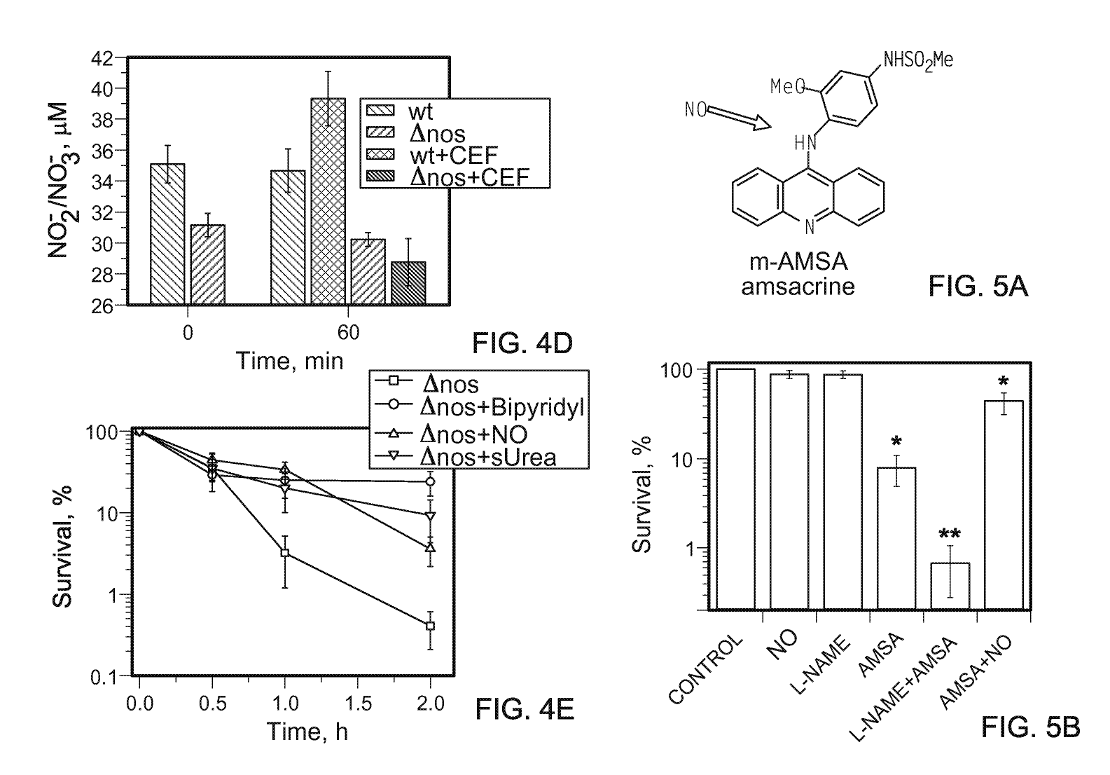

Since NO-mediated protection provides Bacilli and Staphylococci with an important survival advantage, it is likely to be a general defense strategy. Indeed, fungi that produce lactam antibiotics share the same soil niche with Bacilli and Staphylococci. Nine lactams were identified in the phenotypic screen (FIG. 1A), demonstrating that endogenous NO effectively diminishes lactam toxicity against B.subtilis. Notably, a representative lactam, cefuroxime (FIG. 4A), inhibits growth of nos-deficient S.aureus cells to a much greater extent than that of a wild type pathogen (FIG. 4B), suggesting that NO-mediated lactam resistance is not limited to Bacilli, but is likely a general defense mechanism of all bacteria that possess bNOS.

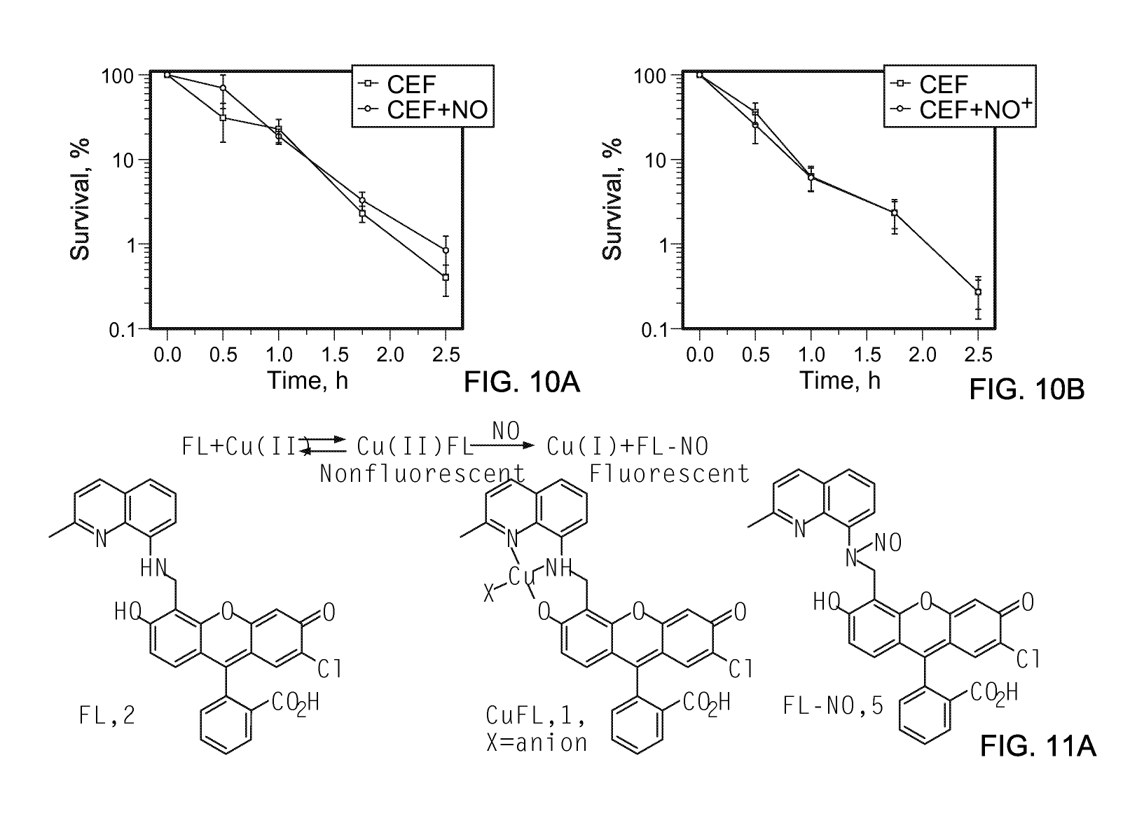

A major target for lactams is cell wall biosynthesis. However, it was shown recently that one of the mechanisms by which ampicillin kills E. coli is by inducing ROS. This ROS-mediated bactericidal effect could be abolished by addition of the iron chelator bipyridyl or the ROS scavenger thiourea (11). Because NO/NO⁺ protect Bacilli against oxidative stress (FIG. 1B) (5, 6), and they do not react with lactams directly (FIG. 10A-B), it is reasonable to propose that NOS activity renders bacteria resistant to lactams by suppressing oxidative stress in a similar, general manner, as demonstrated for ACR and PYO (FIG. 1B). Indeed, cefuroxime kills nos deficient B.subtilis and B.anthracis much more efficiently than wt cells (FIG. 4C). Moreover, pretreatment with exogenous NO temporary protects cells against CEF toxicity (FIG. 4E). Similar protection can be achieved by addition of the iron chelator bipyridyl or the radical scavenger thiourea (FIG. 4E), indicating that CEF causes oxidative stress in B.subtilis, whereas NO protects against it.

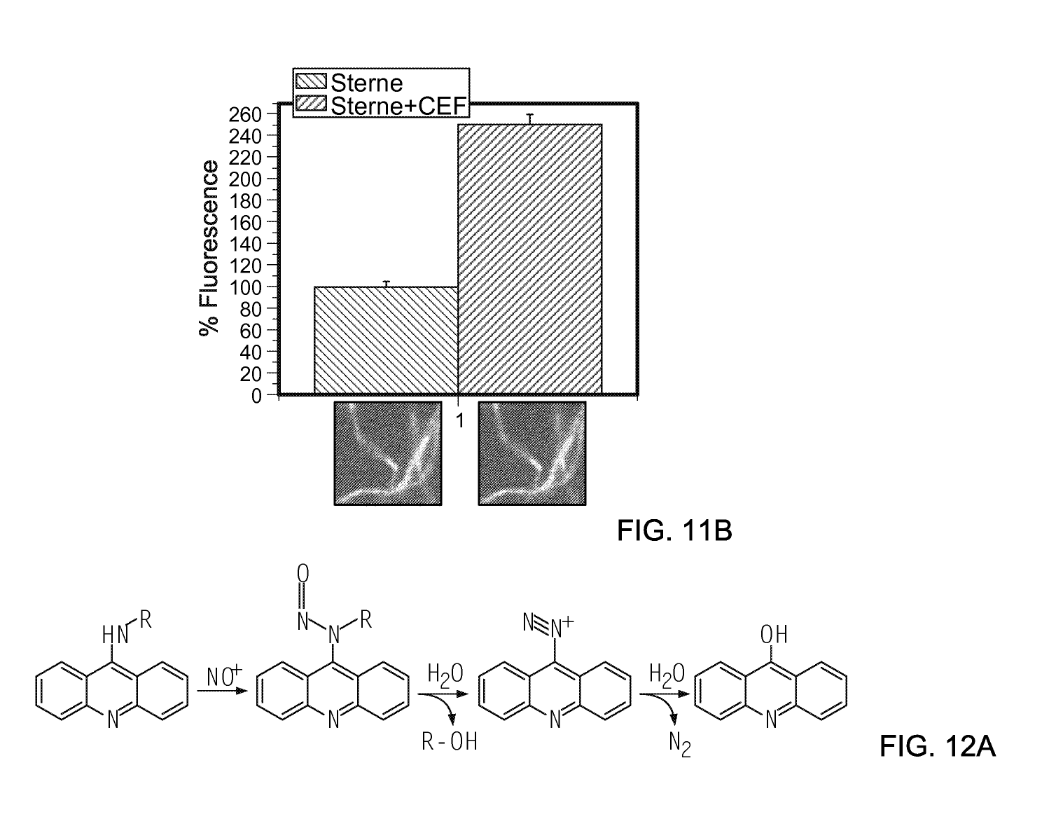

Interestingly, the CEF challenge resulted in increase of the end products of NO oxidation (nitrite/nitrate) in the growing culture of the wt B. subtilis, but not in the Δnos cells (FIG. 4D), indicating that the antibiotic stimulated bNOS activity. To corroborate this result and monitor NO production in vivo directly, a highly specific Cu(II)-based fluorescent NO sensor, CuFL, was utilized (30). This sensor is cell-permeable and allows for NO detection in live cells in real time (4, 6). As shown in FIG. 11B, the antibiotic greatly stimulated NO production in B.anthracis. Since bNOS gene expression was not affected by the antibiotic, these results demonstrate that the enzyme, itself, was activated by the antibiotic treatment. Because bNOS does not have its own reductase domain, it must use cellular redox partners for NO production (4). Direct bNOS activation by antibiotics, therefore, could be due to the accumulation of ROS, which become a part of the feedback loop by serving as electron donors for arginine oxidation by bNOS (FIG. 1B).

The magnitude of bNOS protection against different antibiotics may not be as dramatic as that of specialized antibiotic-resistance gene products. Instead, however, it is remarkably versatile. By analogy with innate immunity, which is less specific than adaptive immunity, the broad protection by bNOS should afford bacteria a tremendous survival advantage in highly competitive environments, such as soil, where bacteria may encounter many different antibiotics. Such a broad spectrum of protection is achieved by two major mechanisms: (1) direct detoxification of a toxic compound, and (2) alleviation of the oxidative stress imposed by many antimicrobials. The latter is mediated by three processes: interruption of the Fenton reaction, direct catalase activation (5), and activation of SOD expression (FIGS. 1B and 3F).

### Endogenous NO is a Universal Detoxifier.

The results disclosed herein suggest that the detoxification function of NOS has been conserved during evolution Akin to bacterial communities that constantly expose each other to toxins, mammalian cells must cope with the toxic products generated by their own metabolism, by infecting pathogens, or present in the environment. It is thus tempting to speculate that eukaryotic NOS, like its bNOS ancestor, has been exploited throughout evolution for detoxification. To substantiate this hypothesis, the inventors examined the role of NO in protecting hepatocytes from a representative cytotoxic compound, AMSAcrine- a clinically approved anticancer drug (31, 32) (FIG. 5A). AMSAcrine is an acridine derivative that can be detoxified by a mechanism similar to NO detoxification of ACR that the inventors described (FIG. 12A). Hepatocytes were examined because the liver is the principle organ in which most chemicals and toxins are normally metabolized and/or detoxified. Moreover, liver cells express iNOS, the inducible form of NOS that generates large amounts of NO (33, 34). As shown in FIG. 5B, preincubation of AMSAcrine with exogenous NO reduced its toxicity to a level that allowed 5.5 fold greater cell survival than that of AMSAcrine alone. Furthermore, inhibition of cellular NOS by the specific inhibitor L-NAME resulted in an increased sensitization of hepatocytes to the drug, indicating that endogenous NO generated by liver NOS is directly involved in AMSAcrine detoxification (FIG. 5B).

AMSAcrine is bright yellow. It has a characteristic absorption peak at 435 nM, which is decreased and shifted upon reaction with NO⁺ (FIG. 5C). The changes in this absorption at 435 nM were used to observe the steady, NOS-dependent degradation of AMSAcrine by hepatocytes; the NOS inhibitors, L-NAME or L-NIL, greatly compromised this AMSAcrine degradation (FIG. 5D). Whereas L-NAME inhibits all NOS isoforms, L-NIL is specific for iNOS, thus directly implicating iNOS as a principle detoxifier of AMSAcrine. This observation suggests that drug detoxification may be a newly recognized, major function of iNOS.

### Clinical Implications.

The results of this study have important clinical implications. The role of NOS in controlling some chronic bacterial infections has been clearly demonstrated in recent years (35, 36). However, since endogenous NO compromises the effectiveness of many standard antibiotics, NOS inhibition should be considered as an adjuvant treatment for acute antibacterial therapies. Moreover, some notorious pathogens such as B.anthracis and S.aureus possess bNOS, which protects them not only against antibiotics, but also against immune attack (6). Therefore, specific inhibition of bNOS in these organisms could be an effective antibacterial intervention. bNOS has several unique features that distinguish it from its mammalian NOS counterparts, suggesting that bNOS-specific inhibitors could be designed. Indeed, some potent bNOS inhibitors have already been described (37). The present observation that NO effectively neutralizes a major toxin produced by P.aeruginosa suggests that NO can be administered therapeutically to combat lung infections of cystic fibrosis patients. Indeed, it has been shown that the amount of exhaled NO is decreased in individuals with cystic fibrosis, which negatively affected their condition (38). Moreover, stimulation of NO synthesis by L-arginine inhalation improved their symptoms (38). Finally, the ability of NO to detoxify therapeutic drugs (FIG. 5 and 12B) suggests that by inhibiting NOS it may be possible to decrease the effective concentrations of toxic drugs, thereby diminishing their damaging side effects. NOS activity should, therefore, be considered in the design and use of chemotherapeutics and other acutely administered drugs.

### REFERENCES

1. K. Pant, A. M. Bilwes, S. Adak, D. J. Stuehr, B. R. Crane, Biochemistry 41, 11071-9 (Sep. 17, 2002).
2. K. Pant, B. R. Crane, Biochemistry 45, 2537-44 (Feb. 28, 2006).
3. D. J. Stuehr, Biochim Biophys Acta 1411, 217-30 (May 5, 1999).
4. I. Gusarov et al., J Biol Chem 283, 13140-7 (May 9, 2008).
5. I. Gusarov, E. Nudler, Proc Natl Acad Sci USA 102, 13855-60 (Sep. 27, 2005).
6. K. Shatalin et al., Proc Natl Acad Sci USA 105, 1009-13 (Jan. 22, 2008).
7. E. G. Johnson et al., Chem Biol 15, 43-50 (Jan. 2008).
8. T. Andoh, R. Ishida, Biochim Biophys Acta 1400, 155-71 (Oct. 1, 1998).
9. D. J. Dwyer, M. A. Kohanski, B. Hayete, J. J. Collins, Mol Syst Biol 3, 91 (2007).
10. M. Wainwright, J Antimicrob Chemother 47, 1-13 (January 2001).
11. M. A. Kohanski, D. J. Dwyer, B. Hayete, C. A. Lawrence, J. J. Collins, Cell 130, 797-810 (Sep. 7, 2007).
12. M. A. Kohanski, D. J. Dwyer, J. Wierzbowski, G. Cottarel, J. J. Collins, Cell 135, 679-90 (Nov. 14, 2008).
13. M. Wainwright, J. E. Kristiansen, Int J Antimicrob Agents 22, 479-86 (November 2003).
14. A. Nedospasov, R. Rafikov, N. Beda, E. Nudler, Proc Natl Acad Sci USA 97, 13543-8 (Dec. 5, 2000).
15. N. R. Asad, A. C. Leitao, J Bacteriol 173, 2562-8 (April 1991).
16. S. Moncada, R. M. Palmer, E. A. Higgs, Pharmacol Rev 43, 109-42 (June 1991).
17. J. S. Stamler, D. J. Singel, J. Loscalzo, Science 258, 1898-902 (Dec. 18, 1992).
18. C. N. Cruickshank, E. J. Lowbury, Br J Exp Pathol 34, 583-7 (December 1953).
19. D. E. Stewart-Tull, A. V. Armstrong, J Med Microbiol 5, 67-73 (February 1972).
20. R. Wilson et al., J Clin Invest 79, 221-9 (January 1987).
21. G. W. Lau, D. J. Hassett, H. Ran, F. Kong, Trends Mol Med 10, 599-606 (December 2004).
22. R. Wilson et al., Infect Immun 56, 2515-7 (September 1988).
23. S. S. Baron, J. J. Rowe, Antimicrob Agents Chemother 20, 814-20 (December 1981).
24. H. M. Hassan, I. Fridovich, J Bacteriol 141, 156-63 (January 1980).
25. L. E. Dietrich, A. Price-Whelan, A. Petersen, M. Whiteley, D. K. Newman, Mol Microbiol 61, 1308-21 (September 2006).
26. L. G. Rahme et al., Science 268, 1899-902 (Jun. 30, 1995).
27. D. V. Vukomanovic et al., Biochem J322 (Pt 1), 25-9 (Feb. 15, 1997).
28. T. Inaoka, Y. Matsumura, T. Tsuchido, J Bacteriol 180, 3697-703 (July 1998).
29. T. Inaoka, Y. Matsumura, T. Tsuchido, J Bacteriol 181, 1939-43 (March 1999).
30. M. H. Lim, D. Xu, S. J. Lippard, Nat Chem Biol 2, 375-80 (July 2006).
31. P. A. Cassileth, R. P. Gale, Leuk Res 10, 1257-65 (1986).
32. L. P. Miller et al., Cancer 67, 2235-40 (May 1, 1991).
33. D. A. Geller et al., J Surg Res 55, 427-32 (October 1993).
34. B. S. Taylor, L. H. Alarcon, T. R. Billiar, Biochemistry (Mosc) 63, 766-81 (July 1998).
35. F. C. Fang, Nitric Oxide and Infection (Kluwer Acad./Plenum Publishers, NY, 1999).
36. C. Nathan, M. U. Shiloh, Proc Natl Acad Sci USA 97, 8841-8 (Aug. 1, 2000).
37. I. Salard et al., J Inorg Biochem 100, 2024-33 (December 2006).
38. H. Grasemann, F. Kurtz, F. Ratjen, Am J Respir Crit Care Med 174, 208-12 (Jul. 15, 2006).
39. K. Kobayashi, J Bacteriol 189, 4920-31 (July 2007)
40. J. M. Farrow, 3^{rd}, E. C. Pesci, J Bacteriol 189, 3425-33 (May, 2007)
41. S. H. Fisher, A. L. Sonenshein, annu Rev Microbiol 45, 107-35 (1991)
42. K. Shatalin, A. Neyfakh, FEMS Microbiol Lett 245, 315-19 (April, 2005)

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

What is claimed is:

1. A method for enhancing efficacy of an antimicrobial, anti-protozoa or anti-cancer treatment in a subject, wherein said treatment comprises administering to the subject a compound which becomes inactivated by NO or natural products of NO oxidation in vivo or becomes less effective due to NO action, said method comprising co-administering said compound with an inhibitor of endogenous NO production or NO scavenger.

2. The method of claim 1, wherein the compound which becomes inactivated by NO or natural products of NO oxidation in vivo or becomes less effective due to NO action and the inhibitor of endogenous NO production or NO scavenger are administered simultaneously.

3. The method of claim 1, wherein the compound which becomes inactivated by NO or natural products of NO oxidation in vivo or becomes less effective due to NO action and the inhibitor of endogenous NO production or NO scavenger are administered sequentially.

4. The method of claim 1, wherein the compound which becomes inactivated by NO or natural products of NO oxidation in vivo or becomes less effective due to NO action and the inhibitor of endogenous NO production or NO scavenger are administered in the same composition.

5. The method of claim 1, wherein the compound which becomes inactivated by NO or natural products of NO oxidation in vivo or becomes less effective due to NO action and the inhibitor of endogenous NO production or NO scavenger are administered in different compositions.

6. The method of claim 1, wherein the inhibitor of endogenous NO production is selected from the group consisting of L-arginine, N G -monomethyl-L-arginine (NMMA), N G -nitro-L-arginine methyl ester (NAME), N G -nitro-L-arginine (NNA), N G -amino-L-arginine (NAA), N G,N G -dimethylarginine (asymmetric dimethylarginine, called ADMA), L-Thiocitrulline, S-methyl-L-Thiocitrulline, diphenyleneiodonium chloride, 2-(4-carboxyphenyl)-4,4,5,5-tetramethylimidazoline-1-oxy 3-oxide, 7-nitroindazole, N(5)-(1-iminoethyl)-L-ornithine, aminoguanidine, canavanine, ebselen, S-methyl-L-citrulline, S-methylisourea, and 2-mercaptoethylguanidine.

7. The method of claim 1, wherein the inhibitor of endogenous NO production is an iNOS-specific inhibitor.

8. The method of claim 1, wherein the NO scavenger is selected from the group consisting of non-heme iron-containing peptides, non-heme iron-containing proteins, porphyrins, metalloporphyrins, dithiocarbamates, dimercaptosuccinic acid, phenanthroline, desferrioxamine, pyridoxal isonicotinoyl hydrazone (PIH), 1,2-dimethyl-3hydroxypyrid-4-one (L1), [+] 1,2-bis (3,5-dioxopiperazine-1yl)propane (ICRF-187), and 2-(4-carboxyphenyl)-4,5-dihydro-4,4,5,5-tetramethyl-1H-imidazolyl-1-oxy-3-oxide (Carboxy-PTIO).

9. The method of claim 1, wherein the NO scavenger is a perfluorocarbon emulsion.

10. The method of claim 1, wherein the compound which becomes inactivated by NO or natural products of NO oxidation in vivo or becomes less effective due to NO action is selected from the compounds disclosed in FIGS. 1A and 6A-B and Table 1.

11. The method of claim 10, wherein the compound is selected from the group consisting of 5-Chloro-7-iodo-8-hydroxyquinoline, 8-Hydroxyquinoline, 8-Hydroxy-5-nitroquinoline, Novobiocin, Acriflavine, 9-Aminoacridine, Prochlorperazine, Chlorpromazine, Prochlorperazine, Penimepicycline, Sisomicin, Gentamicin, Cephaloridine, 7-Aminocephalosporanic acid, Cefotaxime, Cefuroxime, Ampicillin, Moxalactam, 6-Aminopenicillanic acid, Amoxicillin, Azlocillin, Proflavine, Panflavine, Planacrine, Gonoflavin, Trypaflavin, Diflavine, Flavicid, Ethacridine (Rivanol), Aminacrine, 3-Amino-10-methyl-6-haloacridinium, 3-Nitro-9-aminoacridine, 9-Amino-2,3-dimethoxy-6-nitroacridine-10-oxides, and Salacrin.

12. The method of claim 1, wherein the compound which becomes inactivated by NO or natural products of NO oxidation in vivo or becomes less effective due to NO action is selected from the compounds disclosed in FIG. 12B and Tables 2-3.

13. The method of claim 12, wherein the compound is an acridine derivative selected from the group consisting of topoisomerase inhibitors, acridine-platinum conjugates, acridine-alkylating agents, telomerase inhibitors, and DNA crosslinking agents.

14. The method of claim 12, wherein the compound is selected from the group consisting of Doxorubicin, Daunorubicin, Mitoxantrone, Actinomycin D, Mithramycin A, Mitomycin C, Bleomycin, Vincristine, Vinorelbine, Paclitaxel, Docetaxel, Irinotecan, Topotecan, and Fumitremorgin C.

15. The method of claim 1, wherein the compound which becomes inactivated by NO or natural products of NO oxidation in vivo or becomes less effective due to NO action is selected from the compounds disclosed in FIG. 12B and Table 4.

16. The method of claim 15, wherein the compound is Pyronaridine or Amodiaquine.

17. The method of claim 1, wherein the treatment is directed against an infection by S.aureus or B. anthracis.

18. The method of claim 1, wherein the treatment is directed against an infection causing pneumonia or endocarditis.

19. The method of claim 1, wherein the treatment is directed against a malarial infection.

20. A method for decreasing an effective concentration of a drug used in an antibacterial, anti-protozoa or chemotherapeutic treatment, wherein said drug becomes inactivated by NO or natural products of NO oxidation in vivo or becomes less effective due to NO action, said method comprising co-administering said drug with an inhibitor of endogenous NO production or NO scavenger.

21. The method of claim 20, wherein the drug and the inhibitor of endogenous NO production or NO scavenger are administered simultaneously.

22. The method of claim 20, wherein the drug and the inhibitor of endogenous NO production or NO scavenger are administered sequentially.

23. The method of claim 20, wherein the drug and the inhibitor of endogenous NO production or NO scavenger are administered in the same composition.

24. The method of claim 20, wherein the drug and the inhibitor of endogenous NO production or NO scavenger are administered in different compositions.

25. The method of claim 20, wherein the inhibitor of endogenous NO production is selected from the group consisting of L-arginine, N G -monomethyl-L-arginine (NMMA), N G -nitro-L-arginine methyl ester (NAME), N G -nitro-L-arginine (NNA), N G -amino-L-arginine (NAA), N G,N G -dimethylarginine (asymmetric dimethylarginine, called ADMA), L-Thiocitrulline, S-methyl-L-Thiocitrulline, diphenyleneiodonium chloride, 2-(4-carboxyphenyl)-4,4,5,5-tetramethylimidazoline-1-oxy 3-oxide, 7-nitroindazole, N(5)-(1-iminoethyl)-L-ornithine, aminoguanidine, canavanine, ebselen, S-methyl-L-citrulline, S-methylisourea, and 2-mercaptoethylguanidine.

26. The method of claim 20, wherein the inhibitor of endogenous NO production is an iNOS-specific inhibitor.

27. The method of claim 20, wherein the NO scavenger is selected from the group consisting of non-heme iron-containing peptides, non-heme iron-containing proteins, porphyrins, metalloporphyrins, dithiocarbamates, dimercaptosuccinic acid, phenanthroline, desferrioxamine, pyridoxal isonicotinoyl hydrazone (PIH), 1,2-dimethyl-3hydroxypyrid-4-one (L1), [+] 1,2-bis (3,5-dioxopiperazine-1yl)propane (ICRF-187), and 2-(4-carboxyphenyl)-4,5-dihydro-4,4,5,5-tetramethyl-1H-imidazolyl-1-oxy-3-oxide (Carboxy-PTIO).

28. The method of claim 20, wherein the NO scavenger is a perfluorocarbon emulsion.

29. The method of claim 20, wherein the drug is selected from the compounds disclosed in FIGS. 1A, 6A-B, 12B and Tables 1-4.

30. The method of claim 29, wherein the drug is selected from the group consisting of 5-Chloro-7-iodo-8-hydroxyquinoline, 8-Hydroxyquinoline, 8-Hydroxy-5-nitroquinoline, Novobiocin, Acriflavine, 9-Aminoacridine, Prochlorperazine, Chlorpromazine, Prochlorperazine, Penimepicycline, Sisomicin, Gentamicin, Cephaloridine, 7-Aminocephalosporanic acid, Cefotaxime, Cefuroxime, Ampicillin, Moxalactam, 6-Aminopenicillanic acid, Amoxicillin, Azlocillin, Proflavine, Panflavine, Planacrine, Gonoflavin, Trypaflavin, Diflavine, Flavicid, Ethacridine (Rivanol), Aminacrine, 3-Amino-10-methyl-6-haloacridinium, 3-Nitro-9-aminoacridine, 9-Amino-2,3-dimethoxy-6-nitroacridine-10-oxides, and Salacrin.

31. The method of claim 29, wherein the drug is an acridine derivative selected from the group consisting of topoisomerase inhibitors, acridine-platinum conjugates, acridine-alkylating agents, telomerase inhibitors, and DNA crosslinking agents.

32. The method of claim 29, wherein the drug is selected from the group consisting of Doxorubicin, Daunorubicin, Mitoxantrone, Actinomycin D, Mithramycin A, Mitomycin C, Bleomycin, Vincristine, Vinorelbine, Paclitaxel, Docetaxel, Irinotecan, Topotecan, and Fumitremorgin C.

33. The method of claim 29, wherein the drug is Pyronaridine or Amodiaquine.
